(19) United States
(12) Patent Application Publication
(10) Pub. No.: US 2020/0179357 A1
(43) Pub. Date: Jun. 11, 2020

(54) TRPA1 ANTAGONISTS FOR TREATMENT OF DRY EYE, OCULAR PAIN AND INFLAMMATION

(71) Applicant: ALGOMEDIX, INC., Mill Creek, WA (US)

(72) Inventors: Jeffrey M. HERZ, Mill Creek, WA (US); Edward A. KESICKI, Mill Creek, CA (US); Charles P. ZUTA, Mill Creek, WA (US)

(21) Appl. No.: 16/315,113

(22) PCT Filed: Jul. 6, 2017
(86) PCT No.: PCT/US17/40976

§ 371 Date: Jan. 3, 2019

Related U.S. Application Data
(60) Provisional application No. 62/359,122, filed on Jul. 6, 2016.

(51) Int. Cl. A61K 31/444; A61K 9/00; A61K 47/38; A61K 47/10; A61K 9/107; A61K 47/32; A61K 47/26; A61K 47/44; A61K 31/497; A61K 31/501; A61K 31/506; A61P 29/00

(52) U.S. Cl. CPC A61K 31/444; A61K 9/0048; A61K 47/38; A61K 47/10; A61K 9/107; A61K 47/32; A61K 31/7052; A61K 47/44; A61K 31/497; A61K 31/501; A61K 31/506; A61P 29/00; A61K 47/26

(57) ABSTRACT

Stable, clear, ophthalmic formulations and methods of use for an opthalmologically therapeutic effective amount of a TRPA1 antagonist, the formulation comprising buffer system, a viscosity enhancing agent, optionally a preservative, with an opthalmologically acceptable osmolarity and pH, all in an aqueous vehicle. These formulations are effective for treating ocular diseases or conditions caused by, or associated with, or accompanied by ocular pain and inflammatory processes, including, among others, dry eye N disease, uveitis, or any trauma caused by eye surgery or eye injury.

### CROSS-REFERENCE TO RELATED APPLICATIONS

This application is an International Application that claims the priority of U.S. Provisional Application Ser. No. 62,359,122, filed Jul. 6, 2016, titled “TRPA1 ANTAGONISTS FOR TREATMENT OF DRY EYE, OCULAR PAIN, AND INFLAMMATION,” the disclosure of which is incorporated herein by reference in its entirety.

### STATEMENT AS TO RIGHTS TO INVENTIONS MADE UNDER FEDERALLY SPONSORED RESEARCH AND DEVELOPMENT

This invention was made with government support under grant 1R43DA031516, awarded by National Institute on Drug Abuse. The U.S. government has certain rights in the invention.

### FIELD OF THE INVENTION

The invention relates to the treatment of ocular pain, inflammation and symptoms of dry eye disorders. In particular, the invention relates to the use of certain transient receptor potential cation channel, subfamily A, member 1 (TRPA1) inhibitors in the treatment of dry eye disease, ocular pain and ocular inflammation.

### BACKGROUND OF THE INVENTION

Pain is a perceived nociceptive response to local stimuli in the body. The perception of pain at the level of the central nervous system requires the transmission of painful stimuli by peripheral sensory nerve fibers. Upon stimulation of tissue (i.e., thermal, mechanical or chemical), electrochemical signals are transmitted from the sensory nerve endings to the brain where pain is perceived.

The cornea is highly innervated with sensory afferent nerve fibers which transmit various painful stimuli to the central nervous system. Pain conditions involving the eye, therefore, can arise from numerous causes and conditions, such as: foreign body stimulus, inflammation, dry eye syndrome, accidental trauma, surgical procedures and post-surgical recovery. For example, ocular pain can result from photorefractive keratotomy (“PRK”), a vision correcting surgical procedure whereby a laser is used to shape the cornea. This process involves the photoablation of Bowman's membrane and the stromal levels of the cornea. As a result, the denuding of the nerve-containing epithelial layers of the cornea can cause some patients to experience pain following laser surgery until the epithelium regenerates.

Various therapies have been attempted for the alleviation of ocular pain. The use of non-steroidal anti-inflammatory drugs (NSAIDs), such as diclofenac, bromfenac, and ketorolac have been utilized to treat ocular pain and inflammation. These agents inhibit cyclooxygenase (COX)- dependent prostaglandin synthesis. However, some patients have experienced serious complications from use of ocular NSAIDs. Keratitis and ulceration without loss of tissue, corneal and scleral melting, and corneal and scleral perforations are among the more serious complications that have occurred with topical NSAID use. The use of topical NSAIDs also must be avoided in patients in higher risk groups which include patients with sarcoidosis, rosacea, chemical burns, or local radiation around the eye, as examples.

Local anesthetics are another class of pain modulators that relieve pain by directly inhibiting nerve cellular function. One problem with local anesthetic therapy is that the anesthetics exhibit a short duration of action. Another problem with the use of local anesthetics is that their mechanism of actionnon-specific inhibition of many subtypes of voltage-dependent sodium channelscauses block of nerve conduction, which results in loss of sensation and a feeling of “numbness”. Moreover, voltage-dependent sodium channels are also present on other non-neuronal cell types, including smooth muscle, and cardiac muscle.

For treating dry eye and many ocular pain and inflammatory conditions, it is important to provide therapeutic treatment for chronic conditions lasting from many days to many months. For therapeutic treatment of such conditions, it is not safe or practical to numb the corneal surface for such extended periods. Local anesthetics can have the undesired coincident effect of also inhibiting biological functions of other cells, such as fibroblasts and surrounding neural cells. Therefore, even though pain sensations can be abated with local anesthetic treatment, healing and normal function of the tissue may be significantly compromised.

There is a need, therefore, to discover agents which potently and specifically inhibit the transmission of painful stimuli by sensory afferent nerves in the eye, but are without local anesthetic activity or numbing activity, following ocular administration. Administration of TRPA1 antagonists to the eye for treating ocular diseases can provide relief of pain without causing numbness or lack of sensation, thus presenting a solution to this problem.

Furthermore, such pharmacologic agents must be highly specific in the desired activity to inhibit pain without affecting the function of other sensitive normal functions of the eye or ocular tissues, such as maintaining normal ocular pressure and lacrimation, presenting no ocular toxicity, and having no effects on the functions of the posterior segment of the eye, including the retina.

In addition to treating ocular pain, local topical ocular application of local anesthetics has been proposed to reduce or eliminate sensations on the ocular surface to treat the symptoms of dry eye. However, chronic use of topical local anesthetics is thought to seriously delay or prevent regeneration of the epithelium in an injured eye and chronic use may accompanied by toxic side effects. Hence, such use by practitioner is not advised. TRPA1 antagonists can be used to treat dry eye without adverse effects associated with local anesthetics.

Dry eye syndrome or dry eye disease, also referred to as keratoconjunctivitis sicca (hereinafter referred to as dry eye disease), is a common ophthalmological disorder affecting millions of persons each year. The condition is particularly widespread among post-menopausal women because of hormonal changes following the cessation of fertility. Dry eye may afflict an individual with varying severity. In mild cases, a patient may experience burning, stinging, dryness, grittiness, itching and persistent irritation such as is often caused by small bodies lodging between the eye lid and the eye surface. There are often signs of corneal and conjuctival inflammation. In severe cases, vision may be substantially impaired. Other diseases, such as Sjogren's disease and cicatricial pemphigoid, may also lead to dry eye conditions. Transient symptoms of dry eye associated with refractive surgery have been reported to last in some cases from six weeks to six months or more following surgery. Dry eye disease can be diagnosed based on characteristic symptoms and clinical appearance.

Although it appears that dry eye may result from a number of unrelated pathogenic causes, all presentations of the complication share a common effect, that is the breakdown of the pre-ocular tear film, which results in exposure of the ocular surface, dehydration, and cytokine production resulting in many of the symptoms outlined above (Lemp, Report of the National Eye Institute/Industry Workshop on Clinical Trials in Dry Eyes, The CLAO Journal, volume 21, number 4, pages 221-231 (1995)).

Practitioners have taken several approaches to treat the symptoms of dry eye. One common approach has been to supplement and stabilize the ocular tear film using so-called artificial tears instilled throughout the day. Other approaches include the use of ocular inserts that provide a tear substitute or stimulation of endogenous tear production. Examples of the tear substitution approach include the use of buffered, isotonic saline solutions, aqueous solutions comprising water-soluble polymers that render the solutions more viscous and thus less easily shed by the eye.

Although these approaches have met with some success, problems in the treatment of dry eye nevertheless remain, since the use of tear substitutes, while temporarily effective, does not modify the disease process and generally requires repeated applications over extended periods of time (from many weeks to years). The approach of using artificial tears does not directly modulate the activity of the corneal nociceptor nerve fiber endings which are the source of ocular pain signals. Treatment of dry eye disease with artificial tears is not disease modifying since it does not slow or stop progression.

In contrast, in certain aspects of this invention, administering a TRPA1 antagonist to the surface of the eye acts directly to modify the activity of the corneal nerves that are the source of pain and irritation. Thus, the pharmacological treatment using TRPA1 antagonists of this invention is unique since these compounds have the capacity to function as a disease modifying drugs rather than providing only temporary symptomatic relief

Aside from efforts described above, which are directed primarily to the palliative alleviation of symptoms associated with dry eye, methods and compositions directed to treatment of the physiological conditions that cause such symptoms have also been pursued. For example, U.S. Pat. No. 5,041,434 (Lubkin) discloses the use of sex steroids, such as conjugated estrogens, to treat dry eye conditions in post-menopausal women; U.S. Pat. No. 5,290,572 (MacKeen) discloses the use of finely divided calcium ion compositions to stimulate pre-ocular tear film production.

Such efforts to treat the underlying causes of dry eye have focused on treating inflammation of the relevant ocular tissues and meibomian gland dysfunction. The use of various types of agents for such treatment of dry eye patients has been disclosed, including steroids (e.g., U.S. Pat. No. 5,958,912; Marsh et al., Topical non-preserved methylprednisolone therapy for keratoconjunctivitis sicca in Sjogren syndrome, Ophthalmology, 106(4): 811-816 (1999); and Pflugfelder et al., U.S. Pat. No. 6,153,607), cytokine release inhibitors (Yanni, J. M.; et. al. WO 00/03705 A1), cyclosporine A (Tauber, J. Adv. Exp. Med. Biol. 1998, 438 (Lacrimal Gland, Tear Film, and Dry Eye Syndromes 2), 969), and mucosecretatogues, such as 15-HETE (Yanni et. al., U.S. Pat. No. 5,696,166).

Transient receptor potential cation channel, subfamily A, member 1 (TRPA1), is a receptor-ion channel belonging to the transient receptor potential (TRP) family. TRPA1 is a non-selective cation channel that can be activated by endogenous agonists, such as certain cycloprostane prostaglandins, and numerous electrophilic exogenous compounds (e.g. AITC). Activation of TRPA1 leads to an influx of cations and depolarization of the nerve fiber ending, leading to nociceptor nerve fiber signaling and results in pain and inflammation. TRPA1 antagonists, which can alleviate inflammation and pain caused by TRPA1 activation, can selectively block activation of TRPA1 and inhibit activation of certain pain fibers. As discussed herein, TRPA1 antagonists can effectively reduce ocular pain and reduce symptoms of dry eye without causing anesthesia effects on the ocular surface.

Development of an effective ocular formulation for TRPA1 antagonists of the present invention is a challenging endeavor because topical ocular drug delivery is limited by barriers protecting the eye. For TRPA1 modulators of the present invention to be therapeutically effective, the compound must achieve effective concentrations of the drug at critical target sites, such as the cornea, for extended periods of time while minimizing systemic exposure. The ocular bioavailability of the active TRPA1 drug is a major hurdle to overcome with topical application, such as eye drops. The extent of absorption of the ophthalmic drug is severely limited by physiological constraints which include the relatively impermeable corneal barrier. In addition, the physiochemical drug properties of the TRPA1 modulators, such as lipophilicity, solubility, molecular size and shape, possible charge or degree of ionization will affect the route and rate of permeation through the corneal membrane. Hence, the successful design of a formulation for TRPA1 modulators of the present invention requires an integrated knowledge of the unique properties of the drug molecule and the constraints of the ocular route of administration. There have not been any topical formulations of TRPA1 antagonists suitable for the treatment of ocular diseases yet, and no commercially topical TRPA1 antagonist formulation is available. A major challenge in the development of a topical aqueous solutions for ocular delivery is the chemical nature of the compounds of the present invention which includes, but is not limited to, low aqueous water solubility and the need to achieve a high concentration of the compound that can be delivered in a very small volume directly to the eye.

In certain preferred aspects of the present invention, the formulations have been designed for ocular drug delivery to have improved bioavailability and increased duration at the ocular surface by maximizing corneal drug absorption and minimizing precorneal drug loss by including viscosity enhancers and (in certain aspects) including penetration enhancers. Another major concern is achieving efficacy without ocular adverse side effects, such as corneal toxicity, effects on intraocular pressure, and adverse effects on functions of the posterior segment of the eye, including the retina and the optic nerve. It is also important that the ocular formulation not cause blurred vision and should be non-irritative and comfortable when instilled by the patient or doctor. To this end, it is desirable that the TRPA1 antagonist drug formulation be a clear solution and that the viscosity of the solution not provoke lachrymal secretion and reflex blinking for an extended period after instillation. These formulation properties can be created by the appropriate and selective combination of an aqueous buffer, a viscosity enhancer, a penetration enhancer, pH range, osmolarity, and TRPA1 antagonist within a given range of concentrations for each agent or excipient as described within the present invention.

Furthermore, because of physiological and anatomical constraints, only a small fraction of the topically delivered dose of a drug, typically around 1% of the instilled dose, is effectively absorbed. To be clinically effective, the TRPA1 modulator may need to be present at relatively high concentrations in the topical formulation in order for an effective amount to be delivered in a small volume of about 50-100 microliters (e.g., a typical eye drop). This presents further challenges to achieving a suitable ocular formulation for a TRPA1 modulator which must have the proper balance between lipophilic and hydrophilic physical-chemical characteristics to be compatible with an ophthalmic formulation and also have the necessary pharmacological and pharmacokinetic properties to be an effective ocular drug.

In certain preferred aspects, the present invention has achieved the multiple requirements for an ophthalmic drug formulation suitable for delivery to the surface of the eye for the treatment of pain, inflammation, dry eye disease or for uses associated with ocular surgical procedures.

### BRIEF SUMMARY OF THE INVENTION

The invention provides compositions and methods for the treatment of dry eye symptoms, including symptoms of dry eye associated with refractive surgery such as LASIK surgery. In certain aspects of the methods of the invention, certain TRPA1 antagonists are administered to a patient suffering from dry eye syndrome or dry eye disease.

The invention also provides compositions and methods for the treatment of ocular pain and inflammation. In certain aspects of the methods of the invention, TRPA1 antagonists are administered to a patient to prevent or alleviate pain in the eye.

In certain embodiments, the invention provides an ocular formulation, wherein the ocular formulation comprises
(i) a pharmaceutically acceptable carrier comprising
an aqueous buffer,
a viscosity enhancing agent, and
an opthalmologically acceptable inorganic salt or tonicity agent; and
(ii) a pharmaceutically effective amount of a TRPA1 antagonist;
wherein the TRPA1 antagonist is a compound of Formula I:

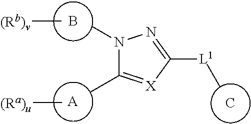

or a pharmaceutically acceptable salt thereof;
   wherein:
A is a cyclic group of Formula Ia:

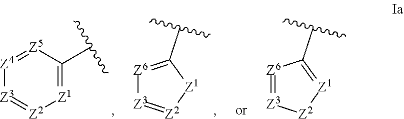

wherein Z¹, Z², Z³, Z⁴, Z⁵, and Z⁶ are each a member selected from the group consisting of N, CH, CR^{a}, and NR^{C}; or, alternatively for Z¹ or Z⁶, the member Z¹ or Z⁶ and X, together with atoms in the rings to which they are attached, form an additional fused, five- to eight-membered cycloalkyl or heterocyclyl ring with from 0 to 4 R^{z} substituents;
with the proviso that at least one member selected from the group consisting of Z², Z³, Z⁴, and Z⁶ is N;
each R^{z} is a member independently selected from the group consisting of halo, C₁-C₃ alkyl, C₁-C₃ haloalkyl, and C₁-C₃ alkoxy; or, alternatively, two R^{z} substituents, together with the carbon atom to which they are attached, join to form an oxo, spirocycloalkyl, or spiroheterocyclyl group;
B is a cyclic group of Formula Ib:

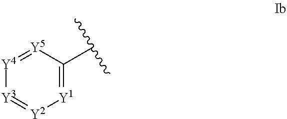

wherein Y¹, Y², Y³, Y⁴, and Y⁵ are each a member independently selected from the group consisting of N, CH, and CR^{b}; or, alternatively, the members —Y²═Y³— or —Y⁴═Y⁵— are combined into a single member selected from the group consisting of NR^{c}, O, and S;

each R^{d} and R^{b} is a member independently selected from the group consisting of cyano, carboxyl, C₁-C₄ alkyl, C₁-C₄hydroxyalkyl, C₃-C₈ cycloalkyl, C₁-C₄ alkoxy, C₁-C₄ haloalkyl, C₁-C₄ haloalkoxy, halo, C₀-C₆ amino, C₁-C₆ amido, C₁-C₄ alkyloxycarbonyl, C₁-C₆ alkylsulfonyl, and hydroxyl; or, alternatively, two adjacent R^{a} or R^{b}, together with the atoms in groups A or B to which they are attached, form an additional fused aryl, heteroaryl, cycloalkyl, or heterocyclyl ring with from 0 to 4 R^{z} substituents;
each R^{c} is a member independently selected from the group consisting of hydrogen, C₁-C₄ alkyl, C₁-C₄ haloalkyl, C₃-C₆ cycloalkyl, (C₃-C₆ cycloalkyl)C₁-C₃alkyl, and C₁-C₇ acyl;
each u is an integer independently selected from 0 to 4;
v is an integer from 0 to 5;
X is N or CR^{d}; or, alternatively, X is CR^{d}, wherein X and the member Z¹, together with atoms in the rings in which they are included, form the additional fused, five- to eight-membered cycloalkyl or heterocyclyl ring with from 0 to 4 R^{z} substituents;
each R^{d} is a member independently selected from the group consisting of hydrogen, halo, cyano, C₁-C₄ alkyl, C₁-C₄ haloalkyl, C₁-C₄ alkoxy, C₁-C₄ haloalkoxy, C₃-C₆ cycloalkyl, and C₃-C₆ cycloalkoxy;
each L¹, L², and L³, if present, is a member independently selected from the group consisting of C═O, C═S, and C═NR^{c};
C is a cyclic group of Formula Ic:

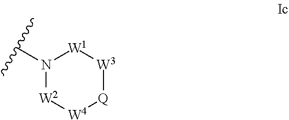

wherein Q is a member selected from the group consisting of C(R^{c})(D), N(E), F, and G; or, alternatively, the members —W³-Q- or —W⁴-Q- join to form a member H; and
wherein W¹, W², W³, and W⁴ are each an independently selected C(R^{f})₂; or, alternatively, the members —W³-Q- or —W⁴-Q- join to form a member H;
R^{e} is a member selected from the group consisting of hydrogen, C₁-C₃ alkyl, and C₁-C₃ fluoroalkyl; or, alternatively, R^{e} and an R^{f} substituent of W¹, W², W³, or W⁴ join to form a —(C(R^{z})ₜ— bridge, wherein t is an integer selected from 2 or 3;
each R^{f} is a member independently selected from the group consisting of hydrogen, C₁-C₃ alkyl, C₁-C₃ fluoroalkyl, and halo; or, alternatively, two adjacent R^{f}, together with the atoms in group C to which they are attached, form an additional aryl, heteroaryl, cycloalkyl, or heterocyclyl fused ring with from 0 to 4 R^{z} substituents; or, alternatively, two geminal R^{f}, together with the atom in group C to which they are attached, form a spirocycloalkyl or spiroheterocyclyl ring with from 0 to 4 R^{z} substituents; or, alternatively, two axial R^{f} substituents of a pair of Wⁿ selected from the group consisting of (W¹ and W²), (W² and W³), and (W³ and W⁴) join to form a —(C(R^{z} )₂)ₜ— bridge; or, alternatively, R^{e} and an R^{f} substituent of W¹, W², W³, or W⁴ join to form a —(C(R^{z})ₜ— bridge;
each t is an integer selected from 2 or 3;
D is a bicyclic group of Formula Id:

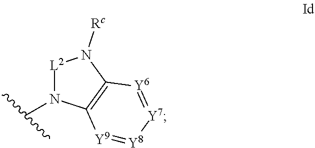

E is a bicyclic group of Formula Ie:

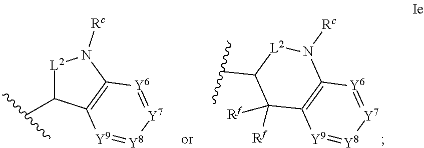

F is a spirocyclic group of Formula If:

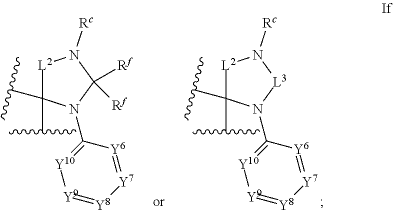

G is a bicyclic spirocyclic group of Formula Ig:

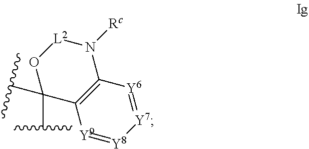

H is a fused group of Formula Ih:

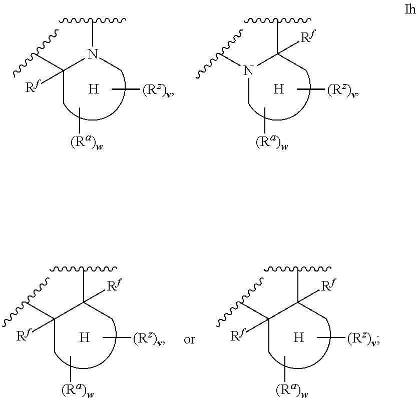

wherein the H ring is a fused, five- to eight-membered cycloalkyl or heterocyclyl ring;
wherein v is an integer from 0 to 4; and
wherein w is an integer from 0 to 2; and
Y⁶, Y⁷, Y⁸, Y⁹, and Y¹⁰, if present, are each a member independently selected from the group consisting of N, CH, and CR^{b}; or, alternatively for Y⁸ and Y⁹, the members —Y⁶═Y⁷— or —Y⁸═Y⁹— are combined into a single member selected from the group consisting of NR^{c}, O, and S.

In certain aspects, the invention provides a method for treating symptoms of dry eye, wherein the method comprises:
administering to an eye of a mammal the ocular formulation as described in the aspects or embodiments herein.

In certain aspects, the invention provides a method for treating ocular pain or ocular inflammation, wherein the method comprises:
administering to an eye of a mammal the ocular formulation as described in the aspects or embodiments herein.

In certain aspects, the invention provides a method for treating pain or inflammation associated with eye surgery, wherein the method comprises:
pre-dosing a patient for a period up to 48 hours before the eye surgery with the ocular formulation as described herein, wherein the formulation is applied to an eye of the patient from one to three times daily; or
applying the formulation post-surgery for about 14 days or until the pain or inflammation has been alleviated, wherein the formulation is applied to the eye of the patient from one to three times daily.

Specific preferred embodiments of the invention will become evident from the following more detailed description of certain preferred embodiments and the claims.

### DETAILED DESCRIPTION OF THE INVENTION

According to the invention, inhibitors of TRPA1 are administered to a patient suffering from dry eye. The compounds suitable for use in the present invention inhibit the activity of TRPA1 by binding to TRPA1 in the eye (e.g., at the cornea) of a patient, thereby reducing the effects of TRPA1 signaling associated with dry eye. The use of TRPA1 antagonists for treating dry eye provides an advantage over current therapies that involve anesthetics, because local treatment of TRPA1 antagonists will not cause loss of ocular sensations associated with anesthesia or have a central analgesic effect. As shown in the Examples herein, TRPA1 antagonists are beneficial in treating various ocular pain states and other conditions that have a neurogenic inflammatory component. In particular, TRPA1 antagonists can inhibit endogenous agonists acting on TRPA1 that provide a major contribution to certain ocular pain conditions. The Examples herein also show that TRPA1 antagonists have significant topical analgesic activity without topical anesthetic activity, thus making them very useful for treating symptoms of dry eye and for treating ocular pain.

### I. Definitions

Unless otherwise stated, the following terms used in this application, including the specification and claims, have the definitions given below. Definition of standard chemistry terms may be found in reference works, including Carey and Sundberg (2007) Advanced Organic Chemistry 5^{th} Ed. Vols. A and B, Springer Science+Business Media LLC, New York.

It must be noted that, as used in the specification and the appended claims, the singular forms “a,” “an” and “the” include plural referents unless the context clearly dictates otherwise. For example, an embodiment including “a TRPA1-modulating compound and an excipient” should be understood to present certain aspects with at least a second TRPA1-modulating compound, at least a second excipient, or both.

Unless otherwise required by context, singular terms used herein shall include pluralities and plural terms shall include the singular.

The practice of the present invention will employ, unless otherwise indicated, conventional methods of synthetic organic chemistry, mass spectroscopy, preparative and analytical methods of chromatography, protein chemistry, biochemistry, recombinant DNA techniques and pharmacology. Conventional methods of organic chemistry include those included in March's Advanced Organic Chemistry: Reactions, Mechanisms, and Structure, 6^{th} Edition, M. B. Smith and J. March, John Wiley & Sons, Inc., Hoboken, N.J., 2007. Although methods and materials similar or equivalent to those described herein can be used in the practice or testing of the present invention, suitable methods and materials are described below. The materials, methods, and examples are illustrative only and not intended to be limiting.

The term “about” as used herein to modify a numerical value indicates a defined range around that value. If “X” were the value, “about X” would generally indicate a value from 0.95× to 1.05×. Any reference to “about X” specifically indicates at least the values X, 0.95X, 0.96X, 0.97X, 0.98X, 0.99X, 1.01X, 1.02X, 1.03X, 1.04X, and 1.05X. Thus, “about X” is intended to teach and provide written description support for a claim limitation of, e.g., “0.98X.” When the quantity “X” only includes whole-integer values (e.g., “X carbons”), “about X” indicates from (X−1) to (X+1). In this case, “about X” as used herein specifically indicates at least the values X, X−1, and X+1.

When “about” is applied to the beginning of a numerical range, it applies to both ends of the range. Thus, “from about 5 to 20%” is equivalent to “from about 5% to about 20%.” When “about” is applied to the first value of a set of values, it applies to all values in that set. Thus, “about 7, 9, or 11%” is equivalent to “about 7%, about 9%, or about 11%.”

The term “acyl” as used herein includes an alkanoyl, aroyl, heterocycloyl, or heteroaroyl group as defined herein. Examples of acyl groups include, but are not limited to, acetyl, benzoyl, and nicotinoyl.

The term “agonist” embraces agents that, e.g., increase, hasten, or activate the expression of a described target protein or bind to, stimulate, increase, open, activate, enhance, increase activation, or up-regulate the activity of one or more proteins (or encoding polynucleotide(s)). As used herein, “agonist” generally includes partial agonists, full agonists, and superagonists (i.e., greater than full agonism). Assays for determining whether a compound “agonizes” or “does not agonize” a protein include, e.g., contacting the protein(s) with the compound and then determining the functional effects on the protein activity or contacting cells expressing protein(s) with the compound and then determining the functional effects on the described target protein activity. One of skill in the art will be able to determine whether an assay is suitable for determining whether a compound agonizes or does not agonize a protein. Samples or assays comprising a TRPA1 target are treated with a test compound and are compared to control samples without the test compound (and to positive control samples activated by exposure to a known TRPA1 agonist) to measure the extent of effect on TRPA1 activity. Control samples (untreated with agonists) are used to establish a baseline activity value. Agonism of the TRPA1 protein or channel is achieved when the activity value increases relative to the untreated control (e.g., increases by 20%, 30%, 40%, 50%, 75%, or 100%, or even more).

The term “antagonist” embraces agents that, e.g., slow or reduce the expression of a described target protein or block, destimulate, decrease, close, deactivate, interfere with, reduce activation, desensitize or down-regulate the activity of one or more proteins (or encoding polynucleotide(s)). As used herein, “antagonist” generally includes partial antagonists and full antagonists. Assays for determining whether a compound “antagonizes” or “does not antagonize” a protein include, e.g., contacting the protein(s) with the test compound and then determining the functional effects on the protein activity or contacting cells expressing protein(s) with the test compound and then determining the functional effects on the described target protein activity. One of skill in the art will be able to determine whether an assay is suitable for determining whether a compound antagonizes or does not antagonize a protein. Samples or assays comprising a TRPA1 target are treated with a putative antagonist and are compared to control samples without the compound (and to control samples activated by a known agonist) to measure the extent of effect on TRPA1 activity. Agonist-activated control samples (untreated with antagonists) are assigned a relative activity value of 100%. Antagonism of the TRPA1 protein or channel activity is achieved when the activity value relative to the agonist-activated control is less than 100% (e.g., 80%, 50%, 40%, 30%, 20%, or 10%, or even lower).

The term “TRPA1 antagonist” and “TRPA1 inhibitor” includes any agent that can inhibit the activity of TRPA1 (i.e. block TRPA1-mediated signaling) at an ophthalmic concentration.

Cells used to determine the agonist or antagonist activity of a compound of the present invention can be cells or cell lines transiently or permanently transfected or transformed with the appropriate nucleic acid encoding TRPA1 or can be cells or cell lines that express TRPA1 from endogenous genes. Typically, the TRPA1 receptor-channel is expressed on the surface of a recombinant host cell such as human embryonic kidney (HEK), CHO, SH-SYSY or COS-7 cells. Alternatively, cells endogenously expressing TRPA1, such as dorsal root ganglion neurons can be isolated from an animal, cultured and used to determine antagonist activity. Examples of cell lines that endogenously express TRPA1 that are suitable for use also include, but are not limited to the human WI-38 cell line, the differentiated human neuroblastoma IMR-32 cell line and the differentiated rat PC12 cell line. The assays described herein can also be performed with cells that express variant TRPA1 proteins. Methods for performing assays to determine if a compound is an agonist or antagonist of the TRPA1 channel are well known in the art. One non-limiting example involves a spectrofluorimetric assay in which cells loaded with a fluorescent dye which is sensitive to intracellular calcium concentrations are subsequently contacted with the compounds of interest to determine their ability to alter intracellular calcium levels. Another method involves testing compounds using an electrophysiological assay, such as patch clamping.

The term “alkanoyl” as used herein embraces an alkyl-C(O)— group wherein the alkyl group is as defined herein. Examples of alkanoyl groups include, but are not limited to, acetyl and propanoyl.

The term “alkyl,” either alone or within other terms such as “haloalkyl” and “alkylamino,” embraces linear or branched radicals having one to about twelve carbon atoms. “Lower alkyl” radicals have one to about six carbon atoms. Examples of such radicals include methyl, ethyl, n-propyl, isopropyl, n-butyl, isobutyl, sec-butyl, tent-butyl, pentyl, isoamyl, hexyl and the like. The term “alkylene” embraces bridging divalent linear and branched alkyl radicals. Examples include methylene, ethylene, propylene, isopropylene and the like.

The term “alkenyl” embraces linear or branched radicals having at least one carbon-carbon double bond of two to about twelve carbon atoms. “Lower alkenyl” embraces radicals having two to about six carbon atoms. Examples of alkenyl radicals include ethenyl, propenyl, allyl, propenyl, butenyl and 4-methylbutenyl. The terms “alkenyl” and “lower alkenyl,” embrace radicals having “cis” and “trans” orientations, or alternatively, “E” and “Z” orientations.

The term “alkoxy” as used herein contemplates an oxygen with a lower alkyl group as a substituent and includes methoxy, ethoxy, butoxy, and the like.

The term “alkynyl” denotes linear or branched radicals having at least one carbon-carbon triple bond and having two to about twelve carbon atoms. “Lower alkynyl” radicals have two to about six carbon atoms. Examples of such radicals include propargyl, butynyl, and the like.

The term “aroyl” as used herein embraces an aryl-CO— group wherein aryl is as defined herein. Examples include, but are not limited to, benzoyl, naphth-1-oyl and naphth-2-oyl.

The term “aryl,” alone or in combination, means a carbocyclic aromatic system comprising one or two rings wherein such rings may be attached together in a fused manner.

The term “aryl” embraces aromatic radicals such as phenyl, naphthyl, indenyl, tetrahydronaphthyl, and indanyl.

The term “comprising” is meant to be open ended, including the indicated component, but not excluding other elements.

The term “cyclic group” means a cycloalkyl, heterocyclyl, aryl, or heteroaryl.

The term “cycloalkyl” includes saturated carbocyclic groups of 3 to 10 carbons. Lower cycloalkyl groups include C₃-C₆ rings. Examples include cyclopentyl, cyclopropyl, and cyclohexyl.

As used herein, “cycloalkylalkyl” embraces an alkyl group wherein the alkyl group includes one or more cycloalkyl substituents (typically one). Examples include, but are not limited to, cyclohexylmethyl, cyclopentylmethyl, and cyclopropylmethyl.

“Formulation,” “pharmaceutical composition,” and “composition” as used interchangeably herein are equivalent terms referring to a composition of matter for pharmaceutical use.

The term “geminal” embraces two or more substituents that are directly attached to the same atom. An example is 3,3-dimethyl substitution on a cyclohexyl or spirocyclohexyl ring.

The term “halo” means halogens such as fluorine, chlorine, bromine or iodine atoms (i.e., fluoro, chloro, bromo, or iodo).

The term “haloalkyl” embraces radicals wherein any one or more of the alkyl carbon atoms is substituted with one or more halo as defined above. Examples include monohaloalkyl, dihaloalkyl and polyhaloalkyl radicals including perhaloalkyl. A monohaloalkyl radical, for one example, may have an iodo, bromo, chloro or fluoro atom within the radical. Dihalo and polyhaloalkyl radicals may have two or more of the same halo atoms or a combination of different halo radicals. “Lower haloalkyl” embraces radicals having 1-6 carbon atoms. Examples of haloalkyl radicals include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. “Perfluoroalkyl” means an alkyl radical having all hydrogen atoms replaced with fluoro atoms. Examples include trifluoromethyl and pentafluoroethyl.

The term “haloalkoxy” means alkoxy radicals wherein any one or more of the alkyl carbon atoms is substituted with one or more halo as defined above. Examples include monohaloalkoxy, dihaloalkoxy and polyhaloalkoxy radicals including perhaloalkoxy. Examples further include difluoromethoxy, trifluoromethoxy and trifluoroethoxy.

The term “heteroaryl” denotes aryl ring systems that contain one or more heteroatoms selected from the group O, N and S, wherein the ring nitrogen and sulfur atom(s) are optionally oxidized, and nitrogen atom(s) are optionally quarternized. Examples include unsaturated 5 to 6 membered heteromonocyclyl group comprising 1 to 4 nitrogen atoms, for example, pyrrolyl, imidazolyl, pyrazolyl, 2-pyridyl, 3-pyridyl, 4-pyridyl, pyrimidyl, pyrazinyl, pyridazinyl, triazolyl; unsaturated 5- to 6-membered heteromonocyclic group comprising an oxygen atom, for example, pyranyl, 2-furyl, 3-furyl, etc.; unsaturated 5 to 6-membered heteromonocyclic group comprising a sulfur atom, for example, 2-thienyl, 3-thienyl, etc.; unsaturated 5- to 6-membered heteromonocyclic group comprising 1 to 2 oxygen atoms and 1 to 3 nitrogen atoms, for example, oxazolyl, isoxazolyl, oxadiazolyl [e.g., 1,2,4-oxadiazolyl, 1,3,4-oxadiazolyl, 1,2,5-oxadiazolyl]; unsaturated 5 to 6-membered heteromonocyclic group comprising 1 to 2 sulfur atoms and 1 to 3 nitrogen atoms, for example, thiazolyl, thiadiazolyl [e.g., 1,2,4-thiadiazolyl, 1,3,4-thiadiazolyl, 1,2,5-thiadiazolyl].

The term “heteroaroyl” embraces a heteroaryl-C(O)— group wherein heteroaryl is as defined herein. Heteroaroyl groups include, but are not limited to, thiophenoyl, nicotinoyl, pyrrol-2-ylcarbonyl, and pyridinoyl.

The term “heterocyclyl” (or “heterocyclo”) embraces saturated, and partially saturated, heteroatom-containing ring radicals, where the heteroatoms may be selected from nitrogen, sulfur and oxygen. Heterocyclic rings comprise monocyclic 6-8 membered rings, as well as 5-16 membered bicyclic ring systems (which can include bridged fused and spiro-fused fused bicyclic ring systems). It does not include rings comprising —O—O—, —O—S— or —S—S— portions.

Examples of saturated heterocyclo groups include saturated 3- to 6-membered heteromonocyclic groups comprising 1 to 4 nitrogen atoms [e.g. pyrrolidinyl, imidazolidinyl, piperidinyl, pyrrolinyl, piperazinyl]; saturated 3 to 6-membered heteromonocyclic group comprising 1 to 2 oxygen atoms and 1 to 3 nitrogen atoms [e.g. morpholinyl]; saturated 3 to 6-membered heteromonocyclic group comprising 1 to 2 sulfur atoms and 1 to 3 nitrogen atoms [e.g., thiazolidinyl]. Examples of partially saturated heterocyclyl radicals include dihydrothienyl, dihydropyranyl, dihydrofuryl, dihydrothiazolyl, and the like.

Particular examples of partially saturated and saturated heterocyclo groups include pyrrolidinyl, imidazolidinyl, piperidinyl, pyrrolinyl, pyrazolidinyl, piperazinyl, morpholinyl, tetrahydropyranyl, thiazolidinyl, dihydrothienyl, 2,3-dihydro-benzo[1,4]dioxanyl, indolinyl, isoindolinyl, dihydrobenzothienyl, dihydrobenzofuryl, isochromanyl, chromanyl, 1,2-dihydroquinolyl, 1,2,3,4-tetrahydro-isoquinolyl, 1,2,3,4-tetrahydro-quinolyl, 2,3,4,4a,9,9a-hexahydro-1H-3-aza-fluorenyl, 5,6,7-trihydro-1,2,4-triazolo[3,4-a]isoquinolyl, 3,4-dihydro-2H-benzo[1,4]oxazinyl, benzo[1,4]dioxanyl, 2,3-dihydro-1H-1λ′-benzo[d]isothiazol-6-yl, dihydropyranyl, dihydrofuryl and dihydrothiazolyl, and the like.

Heterocyclo groups also includes radicals where heterocyclic radicals are fused/condensed with aryl radicals: unsaturated condensed heterocyclic group comprising 1 to 5 nitrogen atoms, for example, indolyl, isoindolyl, indolizinyl, benzimidazolyl, quinolyl, isoquinolyl, indazolyl, benzotriazolyl, tetrazolopyridazinyl [e.g., tetrazolo [1,5-b]pyridazinyl]; unsaturated condensed heterocyclic group comprising 1 to 2 oxygen atoms and 1 to 3 nitrogen atoms [e.g. benzoxazolyl, benzoxadiazolyl]; unsaturated condensed heterocyclic group comprising 1 to 2 sulfur atoms and 1 to 3 nitrogen atoms [e.g., benzothiazolyl, benzothiadiazolyl]; and saturated, partially unsaturated and unsaturated condensed heterocyclic group comprising 1 to 2 oxygen or sulfur atoms [e.g. benzofuryl, benzothienyl, 2,3-dihydro-benzo[1,4]dioxinyl and dihydrobenzofuryl].

The term “heterocycloyl” embraces a heterocyclyl-C(O)— group wherein heterocyclyl is as defined herein. Examples include, but are not limited to, N-methyl prolinoyl and tetrahydrofuranoyl.

The term “hydroxy” or “hydroxyl” embraces —OH.

The term “hydroxyalkyl” embraces radicals wherein any one or more of the alkyl carbon atoms is substituted with one or more hydroxy groups. Examples include hydroxymethyl, 2-hydroxyethyl, and (R)- or (S)-1-hydroxyethyl.

When any two substituent groups or any two instances of the same substituent group are “independently selected” from a list of alternatives, the groups may be the same or different. For example, if R^{a} and R^{b} are independently selected from alkyl, fluoro, amino, and hydroxyalkyl, then a molecule with two R^{a} groups and two R^{b} groups could have all groups be an alkyl group (e.g., four different alkyl groups). Alternatively, the first R^{a} could be alkyl, the second R^{a} could be fluoro, the first R^{b} could be hydroxyalkyl, and the second R^{b} could be amino (or any other substituents taken from the group). Alternatively, both R^{a} and the first R^{b} could be fluoro, while the second R^{b} could be alkyl (i.e., some pairs of substituent groups may be the same, while other pairs may be different). In some embodiments, multiple instances of variables that may be selected from a list of alternatives are independently selected.

The term “interventional procedure” embraces any medical procedure used for diagnosis or treatment that involves incision, puncture, entry into a body cavity, or the use of ionizing, electromagnetic or acoustic energy.

An “ocular formulation” as used herein is a pharmaceutical composition for use in treating a disease or disorder of the eye or for use in ocular surgical or diagnostic procedures.

As used herein, an “ophthalmic concentration” of an active agent is less than 5.0% (w/v).

As used herein, “or” should in general be construed non-exclusively. For example, an embodiment of “a composition comprising A or B” would typically present an aspect with a composition comprising both A and B, and an embodiment of “a method to treat or to prevent” could treat, prevent, or do a combination of both. “Or” should, however, be construed to exclude those aspects presented that cannot be combined without contradiction (e.g., a composition pH that is between 9 and 10 or between 7 and 8).

The term “oxo” as used herein contemplates an oxygen atom attached with a double bond.

The term “periprocedurally” embraces administration of the compound during (intraprocedurally), before (preprocedurally), or after an medical procedure (postprocedurally). In one embodiment, a compound of the present invention is administered (i) preprocedurally or postprocedurally; and (ii) intraprocedurally. The compounds of the present invention are administered to a surgical, diagnostic, or therapeutic procedure site by techniques well known to those of ordinary skill in the art. The compound may be administered periprocedurally, which may include perioperatively (i.e., before, during or after a surgical procedure).

“Pharmaceutically acceptable” means approved or approvable by a regulatory agency of the Federal or state government or listed in the U.S. Pharmacopoeia or other generally recognized pharmacopoeia for use in animals, and more particularly in humans. It can be material which is not biologically or otherwise undesirable, i.e., the material can be administered to an individual without causing any undesirable biological effects or interacting in a deleterious manner with any of the components of the composition in which it is contained.

The term “pharmaceutically acceptable salt” of a compound means a salt that is pharmaceutically acceptable (i.e., non-toxic at the therapeutic dosage) and that possesses the desired pharmacological activity of the parent compound. Such salts include, for example, acid addition salts and base addition salts. Examples of pharmaceutically acceptable salts include inorganic acid addition salts, such as chloride, bromide, sulfate, phosphate, and nitrate; organic acid addition salts, such as acetate, galactarate, propionate, succinate, lactate, glycolate, malate, tartrate, citrate, maleate, fumarate, methanesulfonate, p-toluenesulfonate, and ascorbate; salts with acidic amino acid, such as aspartate and glutamate; alkali metal salts, such as sodium salt and potassium salt; alkaline earth metal salts, such as magnesium salt and calcium salt; ammonium salt; organic base salts, such as tromethamine salt, trimethylamine salt, triethylamine salt, pyridine salt, picoline salt, dicyclohexylamine salt, and N,N′-dibenzylethylenediamine salt; and salts with a basic amino acid, such as lysine salt and arginine salt. The salts may be in some cases hydrates or ethanol solvates.

As used herein, a reference to a composition of formula A, B, C, or a salt thereof embraces A, a salt of A, B, a salt of B, C, or a salt of C.

The term “spirocycloalkyl” embraces a cycloalkyl in which geminal substituents on a carbon atom are replaced to join in forming a 1,1-substituted ring. For example, but without limitation, for a —C(R¹)(R²)— group that was part of a longer carbon chain, if R¹ and R² joined to form a cyclopropyl ring incorporating the carbon to which R¹ and R² were bonded, this would be a spirocycloalkyl group (i.e., spirocyclopropyl).

The term “spiroheterocyclyl” embraces a heterocycloalkyl in which geminal substituents on a carbon atom are replaced to join in forming a 1,1-substituted ring. For example, but without limitation, for a —C(R¹)(R²)— group that was part of a longer carbon chain, if R¹ and R² joined to form a pyrrolidine ring incorporating the carbon to which R¹ and R² were bonded, this would be a spiroheterocyclyl group.

The term “TRPA1 modulator” is a composition that measurably increases or decreases the activity of TRPA1. In certain preferred aspects, the TRPA1 modulator is a TRPA1 antagonist.

Compounds of the present disclosure can exist as stereoisomers, wherein asymmetric or chiral centers are present. Stereoisomers are designated (R) or (S) depending on the configuration of substituents around the chiral carbon atom. The terms (R) and (S) used herein are configurations as defined in IUPAC 1974 Recommendations for Section E, Fundamental Stereochemistry, Pure Appl. Chem., (1976), 45: 13-30, hereby incorporated by reference. The present disclosure contemplates various stereoisomers and mixtures thereof, and these isomers (e.g., a substantially pure (R) or (S) enantiomer of a chiral compound of the present invention) are specifically included within the scope of the present disclosure. Stereoisomers include enantiomers, diastereomers, and mixtures of enantiomers or diastereomers. Individual stereoisomers of compounds of the present disclosure can be prepared synthetically from commercially available starting materials which contain asymmetric or chiral centers or by preparation of racemic mixtures followed by resolution well-known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary or (2) direct separation of the mixture of optical enantiomers on chiral chromatographic columns.

In certain embodiments, the compounds of the invention can be obtained as N-oxides. Conversion of a tertiary amine group (i.e., a compound having the formula R₃N) in a compound of the invention to the corresponding N-oxide (i.e., a compound having the formula R₃N⊕-O\hat{−}) can be conducted chemically according to methods that are known in the art. Conversion of a compound to the N-oxide can also occur after administration to a subject or patient. In certain cases, such conversion is catalyzed enzymatically (e.g., by a cytochrome P450 enzyme). In some instances, the N-oxide can be a metabolite of a tertiary amine present in a compound of the invention. The N-oxide can be an intermediate between the tertiary amine and its N-dealkylated analogs. Depending on the particular compound, an N-oxide can be more active or less active than its parent amine.

Where compounds of the present disclosure include F, I, C or H, the disclosure is not limited to the most common isotopes of these elements. Compounds comprising for example ¹⁸F, ¹⁹, ¹¹C, ¹³C, ¹⁴C, ¹²³I, ¹²⁵I, ³H and ²H are specifically contemplated as being included in the disclosure. A compound of the invention can be radiolabeled according to a number of known techniques. A compound can be radiolabeled, for example, by appending one or more radioisotopes of a halogen (e.g., ¹²⁵1) to an aromatic ring, or by alkylating a nitrogen of a compound of the invention with a group comprising a radioisotope. Radiolabeled compounds can be used to measure binding of the compounds to TRPA1; to detect the compounds in cells, tissues, or organs of a subject to whom the compounds are administered; to enable analysis of compound metabolism; or for radiotherapeutic techniques. Radiolabeled compounds of the invention can also be used as competitive binders in studies for characterizing natural TRPA1 ligands. Isotopes of still other elements can be used in conjunction with the compounds and methods of the invention.

In the Summary of the Invention above, Detailed Description, and the claims below, reference is made to particular features and aspects of the invention, including method steps. The disclosure of the invention in this specification includes all possible combinations of such particular features within the embodiments of the invention disclosed, at least to the extent that such combinations are non-contradictory. For example, if the Detailed Description presents aspects A, B, and C of an embodiment, it is understood that this also discloses particular embodiments including both aspects A and B, both aspects B and C, and both aspects A and C, as well as an embodiment with aspects A, B, and C.

The term “treating” or “treatment” as used herein (and as well understood in the art) means an approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delaying or slowing of disease progression, amelioration or palliation of the disease state, diminishment of the reoccurrence of disease, and remission, whether partial or total and whether detectable or undetectable.

“Treating” and “treatment” as used herein also include prophylactic treatment. Treatment methods comprise administering to a subject a therapeutically effective amount of an active agent. The administering step may consist of a single administration or may comprise a series of administrations. The length of the treatment period depends on a variety of factors, such as the severity of the condition, the age of the patient, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required. For example, the compositions are administered to the subject in an amount and for a duration sufficient to treat the patient.

### II. Formulations

In certain aspects, the invention provides an ocular formulation, wherein the ocular formulation comprises
(i) a pharmaceutically acceptable carrier; and
(ii) a pharmaceutically effective amount of a TRPA1 antagonist;
wherein the TRPA1 antagonist is a compound of Formula I:

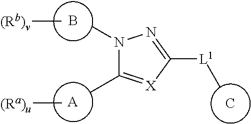

or a pharmaceutically acceptable salt thereof;
   wherein:
A is a cyclic group of Formula Ia:

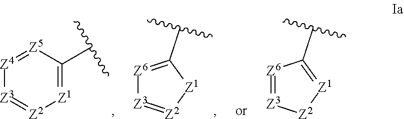

wherein Z¹, Z², Z³, Z⁴, Z⁵, and Z⁶ are each a member selected from the group consisting of N, CH, CR^{a}, and NR^{c}; or, alternatively for Z¹ or Z⁶, the member Z¹ or Z⁶ and X, together with atoms in the rings to which they are attached, form an additional fused, five- to eight-membered cycloalkyl or heterocyclyl ring with from 0 to 4 R^{z} substituents;
with the proviso that at least one member selected from the group consisting of Z², Z³, Z⁴, and Z⁶ is N;
each R^{z} is a member independently selected from the group consisting of halo, C₁-C₃ alkyl, C₁-C₃ haloalkyl, and C₁-C₃ alkoxy; or, alternatively, two R⁷ substituents, together with the carbon atom to which they are attached, join to form an oxo, spirocycloalkyl, or spiroheterocyclyl group;
B is a cyclic group of Formula Ib:

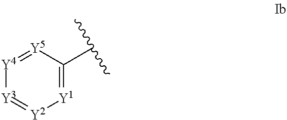

wherein Y¹, Y², Y³, Y⁴, and Y⁵ are each a member independently selected from the group consisting of N, CH, and CR^{b}; or, alternatively, the members —Y²═Y³— or —Y⁴═Y⁵— are combined into a single member selected from the group consisting of NR^{c}, O, and S;
each R^{a} and R^{b} is a member independently selected from the group consisting of cyano, carboxyl, C₁-C₄ alkyl, C₁-C₄hydroxyalkyl, C₃-C₈ cycloalkyl, C₁-C₄ alkoxy, C₁-C₄ haloalkyl, C₁-C₄ haloalkoxy, halo, C₀-C₆ amino, C₁-C₆ amido, C₁-C₄ alkyloxycarbonyl, C₁-C₆ alkylsulfonyl, and hydroxyl; or, alternatively, two adjacent R^{a} or R^{b}, together with the atoms in groups A or B to which they are attached, form an additional fused aryl, heteroaryl, cycloalkyl, or heterocyclyl ring with from 0 to 4 R^{z} substituents;
each R^{c} is a member independently selected from the group consisting of hydrogen, C₁-C₄ alkyl, C₁-C₄ haloalkyl, C₃-C₆ cycloalkyl, (C₃-C₆ cycloalkyl)C₁-C₃alkyl, and C₁-C₇ acyl;
each u is an integer independently selected from 0 to 4;
v is an integer from 0 to 5;
X is N or CR^{d}; or, alternatively, X is CR^{d}, wherein X and the member Z¹, together with atoms in the rings in which they are included, form the additional fused, five- to eight-membered cycloalkyl or heterocyclyl ring with from 0 to 4 R^{z} substituents;
each R^{d} is a member independently selected from the group consisting of hydrogen, halo, cyano, C₁-C₄ alkyl, C₁-C₄ haloalkyl, C₁-C₄ alkoxy, C₁-C₄ haloalkoxy, C₃-C₆ cycloalkyl, and C₃-C₆ cycloalkoxy;
each L¹, L², and L³, if present, is a member independently selected from the group consisting of C═O, C═S, and C═NR^{c};
C is a cyclic group of Formula Ic:

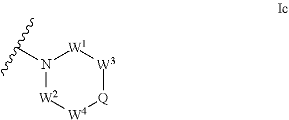

wherein Q is a member selected from the group consisting of C(R^{c})(D), N(E), F, and G; or, alternatively, the members —W³-Q- or —W⁴-Q- join to form a member H; and
wherein W¹, W², W³, and W⁴ are each an independently selected C(R^{f})₂; or, alternatively, the members —W³-Q- or —W⁴-Q- join to form a member H;
R^{e} is a member selected from the group consisting of hydrogen, C₁-C₃ alkyl, and C₁-C₃ fluoroalkyl; or, alternatively, R^{e} and an R^{f} substituent of W¹, W², W³, or W⁴ join to form a —(C(R^{z})₂)ₜ— bridge, wherein t is an integer selected from 2 or 3;
each R^{f} is a member independently selected from the group consisting of hydrogen, C₁-C₃ alkyl, C₁-C₃ fluoroalkyl, and halo; or, alternatively, two adjacent R^{f}, together with the atoms in group C to which they are attached, form an additional aryl, heteroaryl, cycloalkyl, or heterocyclyl fused ring with from 0 to 4 R^{z} substituents; or, alternatively, two geminal R^{f}, together with the atom in group C to which they are attached, form a spirocycloalkyl or spiroheterocyclyl ring with from 0 to 4 R^{z} substituents; or, alternatively, two axial R^{f} substituents of a pair of Wⁿ selected from the group consisting of (W¹ and W²), (W² and W³), and (W³ and W⁴) join to form a —(C(R^{z})₂)ₜ— bridge; or, alternatively, R^{e} and an R^{f} substituent of W¹, W², W³, or W⁴ join to form a —(C(R^{z})₂)ₜ— bridge;
each t is an integer selected from 2 or 3;
D is a bicyclic group of Formula Id:

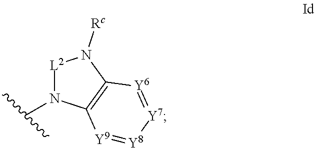

E is a bicyclic group of Formula Ie:

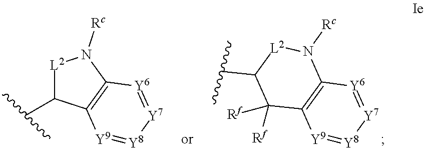

F is a spirocyclic group of Formula If:

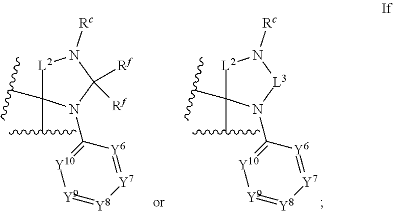

G is a bicyclic spirocyclic group of Formula Ig:

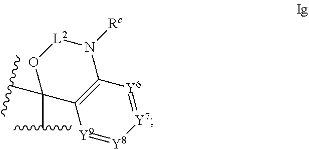

H is a fused group of Formula Ih:

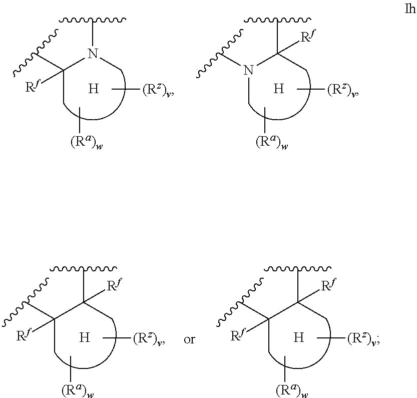

wherein the H ring is a fused, five- to eight-membered cycloalkyl or heterocyclyl ring;
wherein v is an integer from 0 to 4; and
wherein w is an integer from 0 to 2; and
Y⁶, Y⁷, Y⁸, Y⁹, and Y¹⁰, if present, are each a member independently selected from the group consisting of N, CH, and CR^{b}; or, alternatively for Y⁸ and Y⁹, the members Y⁶═Y⁷— or —Y⁸═Y⁹— are combined into a single member selected from the group consisting of NR^{c}, O, and S.

In certain aspects, the invention provides an ocular formulation as described herein, wherein the TRPA1 antagonist is a compound of Formula I:

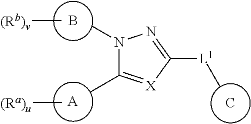

or a pharmaceutically acceptable salt thereof;
   wherein:
A is a cyclic group of Formula Ia:

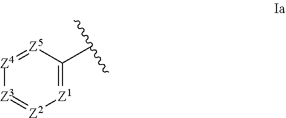

wherein Z¹, Z², Z³, Z⁴, and Z⁵ are each a member selected from the group consisting of N, CH, and CR^{a}; or, alternatively for Z¹, the member Z¹ and X, together with atoms in the rings to which they are attached, form an additional fused, five- to eight-membered cycloalkyl or heterocyclyl ring with from 0 to 4 R^{z} substituents;
with the proviso that at least one member selected from the group consisting of Z², Z³, and Z⁴ is N;
each R^{z} is a member independently selected from the group consisting of halo, C₁-C₃ alkyl, C₁-C₃ haloalkyl, and C₁-C₃ alkoxy; or, alternatively, two R^{z} substituents, together with the carbon atom to which they are attached, join to form an oxo, spirocycloalkyl, or spiroheterocyclyl group;
B is a cyclic group of Formula Ib:

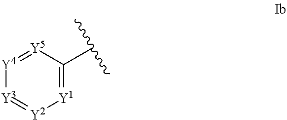

wherein Y¹, Y², Y³, Y⁴, and Y⁵ are each a member independently selected from the group consisting of N, CH, and CR^{b}; or, alternatively, the members —Y²═Y³— or —Y⁴═Y⁵— are combined into a single member selected from the group consisting of NH, NR^{c}, O, and S;
each R^{a} and R^{b} is a member independently selected from the group consisting of cyano, carboxyl, C₁-C₄ alkyl, C₁-C₄hydroxyalkyl, C₃-C₈ cycloalkyl, C₁-C₄ alkoxy, C₁-C₄ haloalkyl, C₁-C₄ haloalkoxy, halo, C₀-C₆ amino, C₁-C₆ amido, C₁-C₄ alkyloxycarbonyl, C₁-C₆ alkylsulfonyl, and hydroxyl; or, alternatively, two adjacent IV or R^{b}, together with the atoms in groups A or B to which they are attached, form an additional fused aryl, heteroaryl, cycloalkyl, or heterocyclyl ring with from 0 to 4 R^{z} substituents;
each R^{c} is a member independently selected from the group consisting of C₁-C₄ alkyl, C₁-C₄ haloalkyl, C₃-C₆ cycloalkyl, (C₃-C₆ cycloalkyl)C₁-C₃alkyl, and C₁-C₇ acyl;
each u is an integer independently selected from 0 to 4;
v is an integer from 0 to 5;
X is N or CR^{d}; or, alternatively, X is CR^{d}, wherein X and the member Z¹, together with atoms in the rings in which they are included, form the additional fused, five- to eight-membered cycloalkyl or heterocyclyl ring with from 0 to 4 R^{z} substituents;
each R^{d} is a member independently selected from the group consisting of hydrogen, halo, cyano, C₁-C₄ alkyl, C₁-C₄ haloalkyl, C₁-C₄ alkoxy, C₁-C₄ haloalkoxy, C₃-C₆ cycloalkyl, and C₃-C₆ cycloalkoxy;
each L¹, L², and L³, if present, is a member independently selected from the group consisting of C═O, C═S, C═NH, and C═NR^{c};
C is a cyclic group of Formula Ic:

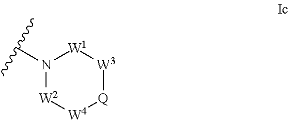

wherein Q is a member selected from the group consisting of C(R^{e})(D), N(E), F, and G; or, alternatively, the members —W³-Q- or —W⁴-Q- join to form a member H; and
wherein W¹, W², W³, and W⁴ are each an independently selected C(R^{f})₂; or, alternatively, the members —W³-Q- or —W⁴-Q- join to form a member H;
R^{e} is a member selected from the group consisting of hydrogen, C₁-C₃ alkyl, and C₁-C₃ fluoroalkyl; or, alternatively, R^{e} and an R^{f} substituent of W¹, W², W³, or W⁴ join to form a —(C(R^{z})₂)ₜ— bridge, wherein t is an integer selected from 2 or 3;
each R^{f} is a member independently selected from the group consisting of hydrogen, C₁-C₃ alkyl, C₁-C₃ fluoroalkyl, and halo; or, alternatively, two adjacent R^{f}, together with the atoms in group C to which they are attached, form an additional aryl, heteroaryl, cycloalkyl, or heterocyclyl fused ring with from 0 to 4 R^{z} substituents; or, alternatively, two geminal R^{f}, together with the atom in group C to which they are attached, form a spirocycloalkyl or spiroheterocyclyl ring with from 0 to 4 R^{z} substituents; or, alternatively, two axial R^{f} substituents of a pair of Wⁿ selected from the group consisting of (W¹ and W²), (W² and W³), and (W³ and W⁴) join to form a —(C(R^{z} )₂)ₜ— bridge; or, alternatively, R^{e} and an R^{f} substituent of W¹, W², W³, or W⁴ join to form a —(C(R²)₂)ₜ— bridge;
each t is an integer selected from 2 or 3;

D is a bicyclic group of Formula Id:

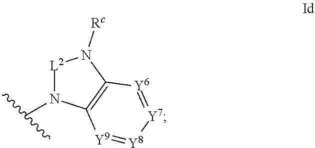

E is a bicyclic group of Formula Ie:

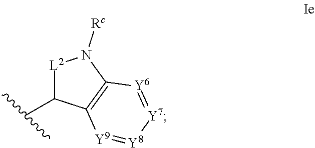

F is a spirocyclic group of Formula If:

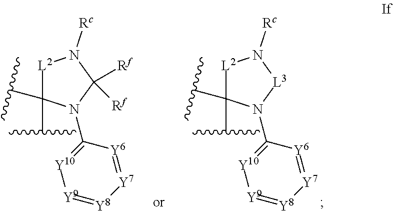

G is a bicyclic spirocyclic group of Formula Ig:

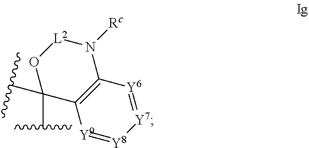

H is a fused group of Formula Ih:

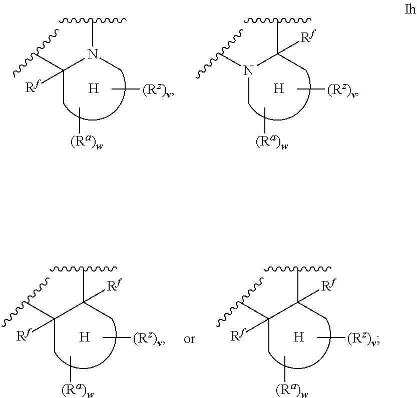

wherein the H ring is a fused, five- to eight-membered cycloalkyl or heterocyclyl ring;
wherein v is an integer from 0 to 4; and
wherein w is an integer from 0 to 2; and
Y⁶, Y⁷, Y⁸, Y⁹, and Y¹⁰, if present, are each a member independently selected from the group consisting of N, CH, and CR^{b}; or, alternatively for Y⁸ and Y⁹, the members —Y⁶═Y⁷— or —Y⁸═Y⁹— are combined into a single member selected from the group consisting of NH, NR^{c}, O, and S.

In certain aspects, the invention provides an ocular formulation as described herein, wherein Q is a member selected from the group consisting of C(R^{e})(D) and F; and
wherein each L² is a member independently selected from the group consisting of C═O and C═S.

In certain aspects, the invention provides an ocular formulation as described herein, The ocular formulation of claim 3, wherein D is a member selected from the group consisting of:

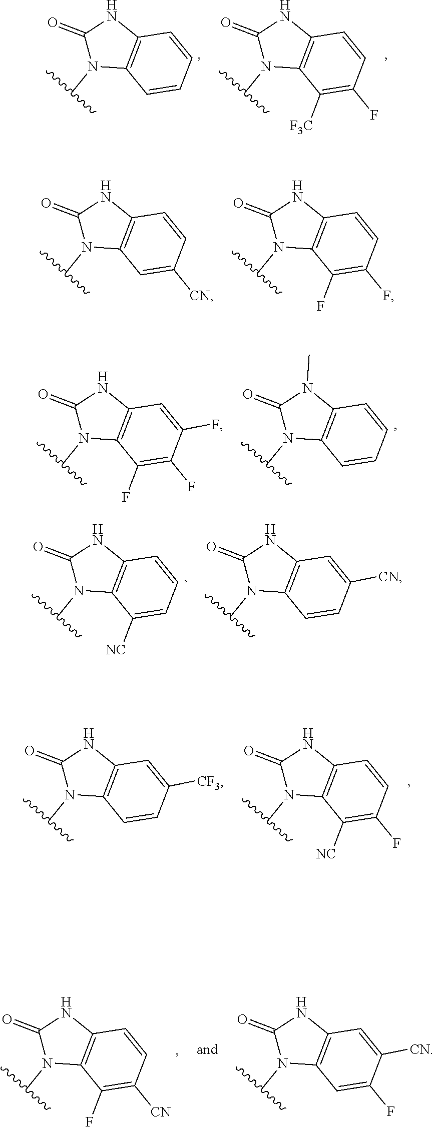

In certain aspects, the invention provides an ocular formulation as described herein, wherein D is a member selected from the group consisting of:

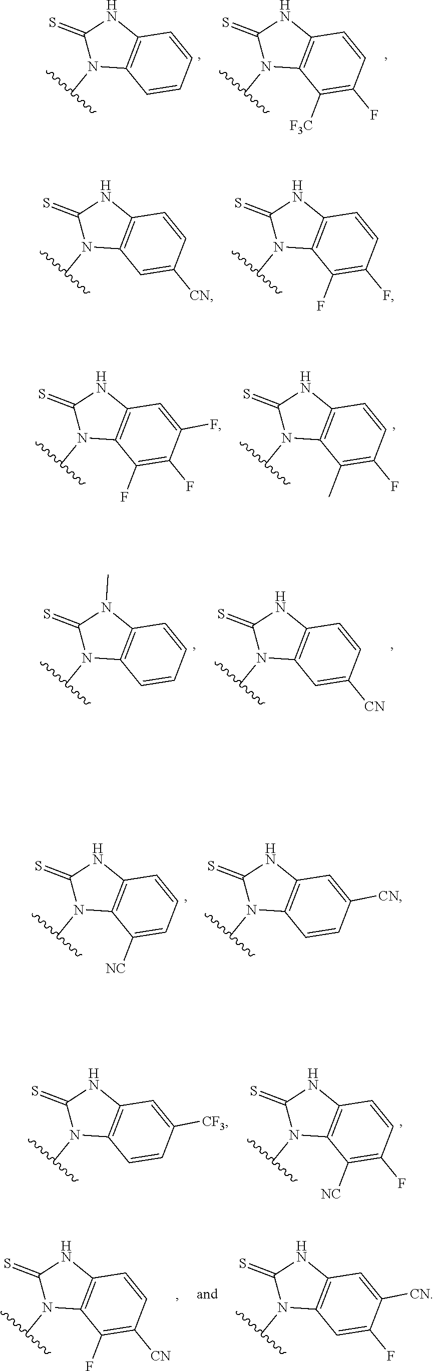

In certain aspects, the invention provides an ocular formulation as described herein, wherein A is a member selected from the group consisting of:

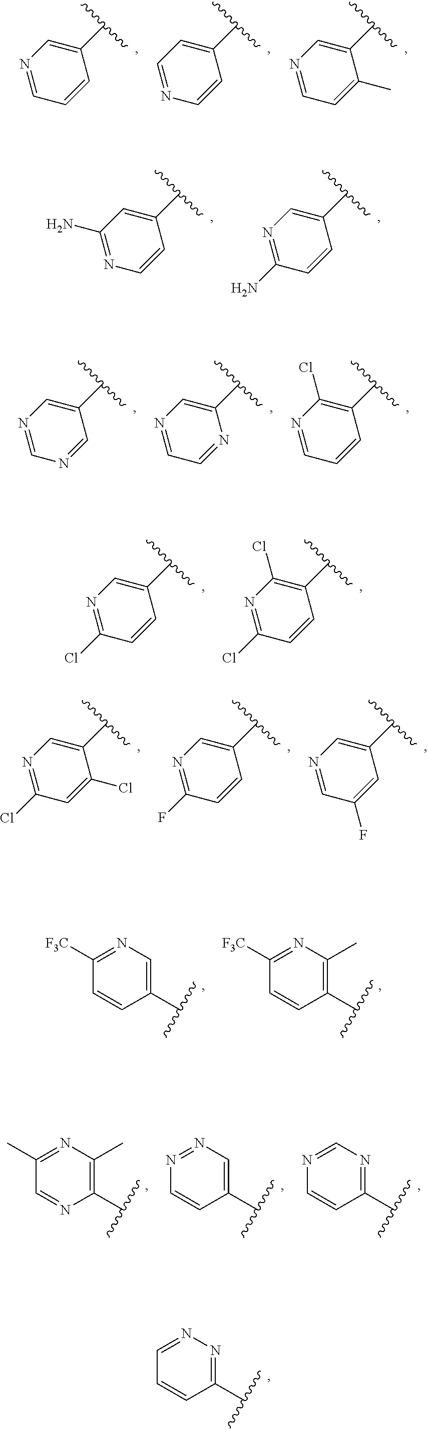

and a salt thereof.

In certain aspects, the invention provides an ocular formulation as described herein, wherein B is a member selected from the group consisting of:

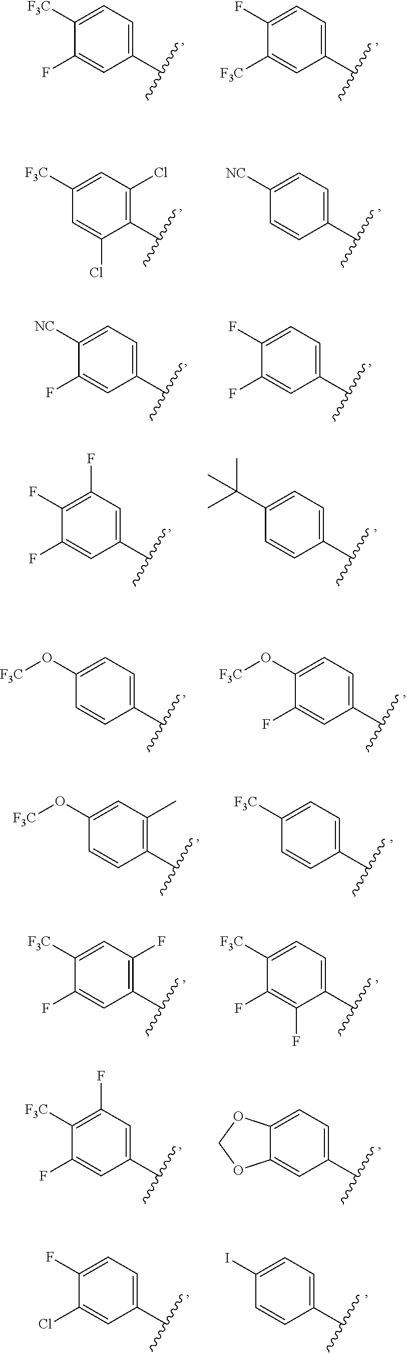

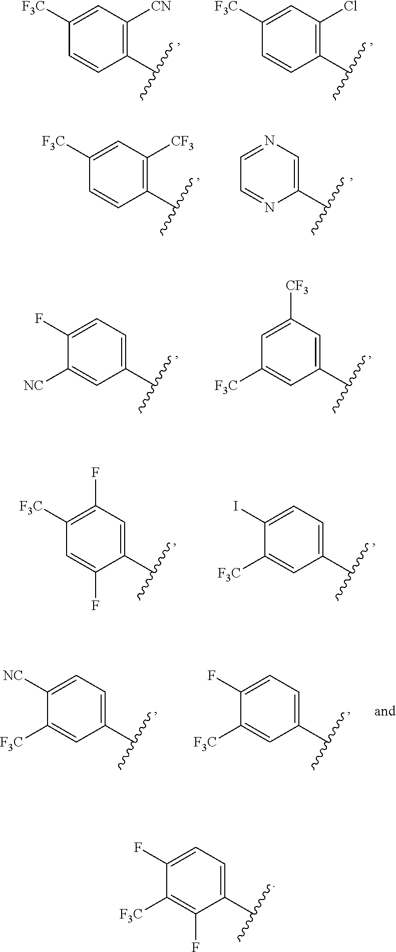

Procedures for preparing representative compounds from the formulae and groups described above are set forth in International Patent Publication No. WO 2015/103060 (PCT/US2014/072291) and its continuation-in-part, the U.S. Non-Provisional Patent Application designated by attorney docket number 097682-1016511-000120US (filed Jul. 6, 2016).

In certain aspects, the invention provides an ocular formulation as described herein, wherein the pharmaceutically effective amount of the TRPA1 antagonist is from 0.001% to 5.0% (w/v). In certain aspects, the pharmaceutically effective amount of the TRPA1 antagonist is from about 0.01% to 2.0% (w/v). In certain aspects, the pharmaceutically effective amount of the TRPA1 antagonist is from about 0.01% to 0.05% (w/v).

Preferably, the compositions administered according to the present invention will be formulated as solutions, suspensions and other dosage forms for topical administration. Aqueous solutions are generally preferred, based on ease of formulation, as well as a patient's ability to easily administer such compositions by means of instilling one to two drops of the solutions in the affected eyes. However, the compositions may also be emulsions, viscous or semi-viscous gels, or other types of solid or semi-solid compositions.

The compositions administered according to the present invention may also include various other ingredients, including but not limited to surfactants, tonicity agents, buffers, preservatives, co-solvents and viscosity enhancing agents.

In certain aspects, the invention provides an ocular formulation as described herein, wherein the carrier comprises an aqueous buffer. In certain aspects, the aqueous buffer is a borate-boric acid system, a citrate-citric acid system, a phosphate-based system, an acetate-acetic acid system, or a combination thereof

In certain aspects, the invention provides an ocular formulation as described herein, wherein the ocular formulation has a pH of from about 6 to 10. In certain aspects, the ocular formulation has a pH of from about 6.3 to 8.0 (e.g., about 6.4, 6.5, 6.6, 6.7, 6.8, 6.9, 7.0, 7.1, 7.2, 7.3, 7.4, 7.5, 7.6, 7.7, 7.8, 7.9, or 8.0). In certain aspects, the pH is from about 6.8 to 7.5 (e.g., about 6.8, 6.85, 6.9, 6.95, 7.0, 7.05, 7.1, 7.15, 7.2, 7.25, 7.3, 7.35, 7.4, 7.45, or 7.5). In certain aspects, the pH is from about 7.0 to 8.0.

An appropriate buffer system (e.g., sodium phosphate, sodium acetate, sodium citrate, bicarbonate, sodium borate or boric acid, TRIS) may be added to the compositions to provide buffering capacity and prevent pH drift under storage conditions and to maintain physiological compatiblity with the ocular surface. The particular concentration will vary, depending on the agent employed. In certain embodiments, the buffer will be chosen to maintain a concentration within the range of 0.5-100 millimolar, 0.5-75 mM, 0.5-50 mM, 0.5-20 mM, or 1-10 mM (e.g., about 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100). In certain preferred aspects, the buffer will be chosen to maintain a target pH within the range of pH 6.0-8.5, preferably 6.5-7.5 (e.g., about 6.5, 6.55, 6.6, 6.65, 6.7, 6.75, 6.8, 6.85, 6.9, 6.95, 7.0, 7.05, 7.1, 7.15, 7.2, 7.25, 7.3, 7.35, 7.4, 7.45, or 7.5). The pH of this solution is measured by standard pH meter at either room temperature or at 37° C.

In certain aspects, the invention provides an ocular formulation as described herein, wherein the ocular formulation comprises a viscosity enhancing agent. In certain aspects, the viscosity enhancing agents are added to the formulation to increase the viscosity of the ophthalmic formulation to improve stability and/or to increase precorneal residence time. Improved precorneal residence time leads to greater transcorneal penetration of the drug into the anterior chamber and an improvement in bioavailability. Exemplary agents include cellulose polymers, carbomer polymers, carbomer derivatives, cellulose derivatives, polyvinyl alcohol, poloxamers, polysaccharides, and the like, as well as mixtures thereof

In addition, enhanced viscosity may result in increased drug bioavailability. Suitable viscosity enhancing reagents may also include hydroxypropylmethylcellulose (HPMC) also known as hypromellose, methylcellulose, carboxymethylcellulose (CMC), polyvinyl alcohol, Polysorbate 80®, polyethylene glycol (PEG) 400 and PEG 300, hydroxyethylcellulose, polyvinylpyrrolidone (povidone) and such class of polymers deemed suitable for effective delivery of the drug. The concentration of these polymers in the solution will range between 0.1%-2.5% to provide viscosity of the formulation within the ranges of 30-150 centipoise (cps) or any specific value within this range, but preferably 70 to 100 cps (e.g., 77 to 88 cps; 70 to 80 cps; 70 to 90 cps; 75 to 90 cps; or 80 to 100 cps). The viscosity of this ophthalmic formulation may be measured according to standard methods known in the art such as by use of viscometer or rheometer and such measurements may be influenced by temperature and shear rate.

In certain aspects, the viscosity enhancing agent is selected from the group consisting of a polysaccharide, a dextran, a polyacrylate, a polyvinyl, and a combination thereof. In certain preferred aspects, the viscosity enhancing agent is selected from the group consisting of 0.1% to 1.5% (w/v) hydroxypropyl methylcellulose (e.g., about 0.1, 0.2, 0.3, 0.4, 0.5, 0.6, 0.7, 0.8, 0.9, 1.0, 1.1, 1.2, 1.3, 1.4, or 1.5% hydroxypropyl methylcellulose); 0.1% to 1.2% (w/v) hydroxyethyl cellulose (e.g., about 0.1, 0.2, 0.3, 0.4, 0.5, 0.6, 0.7, 0.8, 0.9, 1.0, 1.1, or 1.2% hydroxyethyl cellulose); 0.1% to 2.5% (w/v) methylcellulose (e.g., about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, or 2.5% methylcellulose); 0.1% to 1.6% (w/v) polyvinyl alcohol (e.g., about 0.1, 0.2, 0.3, 0.4, 0.5, 0.6, 0.7, 0.8, 0.9, 1.0, 1.1, 1.2, 1.3, 1.4, 1.5, or 1.6% polyvinyl alcohol); 0.1% to 2.0% (w/v) polyvinyl pyrrolidine (e.g., about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, or 2.0% polyvinyl pyrrolidine); 0.1% to 2.1% or 0.1 to 5.0% (w/v) polysorbate (e.g., polyoxyethylene (20) sorbitan monolaurate; polyoxyethylene (20) sorbitan monooleate; polyethylene glycol sorbitan monostearate; polyoxyethylenesorbitan monopalmitate) (e.g., about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0 polysorbate); 0.1% to 2.0% (w/v) polyethylene glycol (e.g., about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, or 2.0% polyethylene glycol); 0.1% to 2.5% (w/v) carboxymethylcellulose (e.g., about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, or 2.5% carboxymethylcellulose); 0.1% to 1.0% (w/v) propylene glycol (e.g., about 0.1, 0.2, 0.3, 0.4, 0.5, 0.6, 0.7, 0.8, 0.9, or 1.0% propylene glycol); 0.1% to 1.0% hyaluronic acid (e.g., about 0.1, 0.2, 0.3, 0.4, 0.5, 0.6, 0.7, 0.8, 0.9, or 1.0% hyaluronic acid); 0.1% to 2.0% (w/v) hyaluronate salt (e.g., about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, or 2.0% hyaluronate salt); and a combination thereof.

In certain aspects, the invention provides an ocular formulation that comprises one or more of the viscosity enhancing agents listed in Table 1.

TABLE 1
Viscosity Enhancing Agents

| | Range of |
|---|---|
| Component | Concentrations |
| Hydroxypropylmethylcellulose | 0.1%-1.5% |
| Hydroxyethylcellulose | 0.1%-1.2% |
| Methylcellulose | 0.1%-2.5% |
| Polyvinyl alchohol | 0.1%-1.6% |
| Polyvinylpyrrolidone | 0.1%-2.0% |
| (povidone) | |
| Polysorbate 80 | 0.1%-5.0% |
| Polyethylene glycol 400 | 0.1%-2.0 % |
| Polyethylene glycol 300 | 0.1%-2.0% |
| Carboxymethlycellulose | 0.1%-2.5% |
| Propyleneglycol | 0.1%-1.0% |
| Hyaluronic acid | 0.1%-1.0% |
| Sodium hyaluronate | 0.1%-2.0% |

In certain aspects, the invention provides an ocular formulation as described herein, wherein the ocular formulation comprises an opthalmologically acceptable inorganic salt. In certain preferred aspects, the opthalmologically acceptable inorganic salt is sodium chloride, potassium chloride, or a combination thereof

In certain aspects, the invention provides an ocular formulation as described herein, wherein the ocular formulation comprises a tonicity agent. In certain aspects, the ocular formulation comprises a tonicity agent that is selected from the group consisting of dextrose, glycerin, mannitol, and a combination thereof

Various tonicity agents may be employed to adjust the tonicity of the composition, preferably to that of natural tears for ophthalmic compositions. For example, sodium chloride, potassium chloride, magnesium chloride, calcium chloride, dextrose, glycerin, propylene glycol, and/or mannitol may be added to the composition to approximate physiological tonicity. Such an amount of tonicity agent will vary, depending on the particular agent to be added. In general, however, the compositions will have a tonicity agent in an amount sufficient to cause the final composition to have an acceptable osmolality for an ophthalmic formulation (generally about 150-450 mOsm/kg, preferably 250-350 mOsm/kg).

In certain aspects, the invention provides an ocular formulation as described herein, wherein the ocular formulation has an osmolality of between about 170 mOSm/kg and 500 mOSm/kg. In certain preferred aspects, the ocular formulation has an osmolality of between about 250 mOSm/kg and 350 mOSm/kg (e.g., about 250, 260, 270, 280, 290, 300, 310, 320, 330, 340, or 350 mOSm/kg). In certain preferred aspects, the ocular formulation has an osmolality of between about 280 mOSm/kg and 310 mOSm/kg (e.g., about 280, 285, 290, 295, 300, 305, or 310 mOSm/kg).

In certain aspects, the invention provides an ocular formulation as described herein, wherein the ocular formulation comprises a non-ionic surfactant. In certain preferred aspects, the non-ionic surfactant is an alkyl aryl polyether alcohol. In certain preferred aspects, the non-ionic surfactant is tyloxapo.

In certain aspects, the non-ionic surfactant is selected from the group including Brij 35, Brij 78, Brij 98, Brij 700, Pluronic F127, Polysorbate 20, Polysorbate 40, Polysorbate 60, Polysorbate 80, Solulan C-24, Span 20, Span, 40, Span 60, Span 80 and Tyloxypol. In certain aspects, the non-ionic surfactant is Brij 35. In certain aspects, the non-ionic surfactant is Brij 78. In certain aspects, the non-ionic surfactant is Brij 98. In certain aspects, the non-ionic surfactant is Brij 700. In certain aspects, the non-ionic surfactant is Pluronic F127. In certain aspects, the non-ionic surfactant is Polysorbate 20. In certain aspects, the non-ionic surfactant is Polysorbate 40. In certain aspects, the non-ionic surfactant is Polysorbate 60. In certain aspects, the non-ionic surfactant is Polysorbate 80. In certain aspects, the non-ionic surfactant is Solulan C-24. In certain aspects, the non-ionic surfactant is Span 20. In certain aspects, the non-ionic surfactant is Span, 40. In certain aspects, the non-ionic surfactant is Span 60. In certain aspects, the non-ionic surfactant is Span 80. In certain aspects, the non-ionic surfactant is Tyloxpol.

Optionally, non-ionic detergents may be added to the formulation to increase absorption or increase the stability of the formulation, which may include: Octoxynol 40, tyloxypol, polyoxyl 40 stearate, and the like.

In certain aspects, the composition includes at least one pharmaceutically acceptable surfactant that is a polyalkylene glycol alkyl ether. The polyalkylene glycol alkyl ether may be present at up to about 2% w/v, such as about 0.1, 0.15, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, or 2.0% w/v. More preferably, the polyalkylene glycol alkyl ether is present at up to about 1.2% w/v, such as about 0.1, 0.15, 0.2, 0.25, 0.35, 0.4, 0.45, 0.5, 0.55, 0.6, 0.65, 0.7, 0.75, 0.8, 0.85, 0.9, 0.95, 1.0, 1.05, 1.1, 1.15, or 1.2% w/v.

In certain aspects, the invention provides an ocular formulation comprising a non-ionic surfactant as described herein, the non-ionic surfactant is from about 0.01% w/v to about 0.5% w/v. In certain aspects, the non-ionic surfactant is from about 0.02% w/v to about 0.3% w/v.

In certain aspects, the invention provides an ocular formulation as described herein, wherein the formulation comprises a second drug that is selected from the group consisting of an antibiotic (e.g., azithromycin), a mydriatic, a local anesthetic, a non-steroidal anti-inflammatory drug, a steroidal anti-inflammatory drug, a drug for treatment of low tear production (e.g., a cyclosporine), an anti-histamine, and a combination thereof. In certain preferred aspects, the second drug is cyclosporine A, azithromycin, or a combination thereof

In certain aspects, the invention provides an ocular formulation as described herein, wherein the ocular formulation is an emulsion.

In certain aspects, the invention provides an ophthalmic emulsion of the TRPA1 agonist compound alone or in combination with cyclosporine A, in combination with emulsifier components, tonicity components, polyelectrolyte components, surfactant components, viscosity inducing components, and acids or bases to adjust pH of the composition. Cyclosporin A or the TRPA1 agonist is solubilized in the hydrophobic component, which is considered as comprising the discontinous phase in the emulsion composition.

In certain aspects, the invention provides an ocular formulation as described herein, wherein the ocular formulation comprises an oil that is selected from the group consisting of an animal oil, a vegetable oil, a fatty acid glyceride, and a combination thereof. In certain aspects, the oil is castor oil, olive oil, or a combination thereof. In certain aspects, the ocular formulation comprises from about 0.625% (w/v) to about 1.5% (w/v) of the oil (e.g., about 0.625, 0.65, 0.70, 0.75, 0.80, 0.85, 0.90, 0.95, 1.0, 1.05, 1.1, 1.15, 1.2, 1.25, 1.3, 1.35, 1.4, 1.45, or 1.5%).

In certain aspects, the invention provides an ocular formulation as described herein, wherein the ocular formulation comprises a carbomer.

In certain preferred aspects, the invention provides an ocular formulation as described herein, wherein the ocular formulation comprises:
a buffer,
a viscosity enhancing agent, and
an opthalmologically acceptable inorganic salt or tonicity agent;

wherein the ocular formulation has an pH of between about 6.0 and 10.0; and

wherein the ocular formulation has an osmolality of between about 170 mOSm/kg and 350 mOSm/kg.

In certain preferred aspects, the invention provides an ocular formulation as described herein, wherein the ocular formulation comprises:
(i) a pharmaceutically acceptable carrier comprising
an aqueous buffer,
a viscosity enhancing agent, and
an opthalmologically acceptable inorganic salt or tonicity agent; and
(ii) a pharmaceutically effective amount of a TRPA1 antagonist as described in any of the aspects and embodiments herein.

In certain preferred aspects, the invention provides an ocular formulation as described herein, wherein the ocular formulation comprises a preservative. In certain aspects, the preservative is selected from the group consisting of a quaternary ammonium compound, hexetidine, an alkyl mercury salt, a paraben, and a combination thereof. In certain aspects the preservative is benzalkonium chloride, methyl paraben, ethyl paraben, hexetidine, a phenyl mercuric salt, chlorobutanol, propylparaben, phenylethyl alcohol, edetate disodium (EDTA), sorbic acid, polyquaternium-1, thiomersal, polyhexamethylene biguanide, sodium perborate, an SofZia buffer system, or a combination thereof

Topical ophthalmic products may also be packaged in multidose form. Preservatives may thus be required to prevent microbial contamination during use. Suitable preservatives include: chlorobutanol, methylparaben, propylparaben, phenylethyl alcohol, edetate disodium, sorbic acid, polyquaternium-1 and benzalkonium chloride (BAK), thiomersal, polyhexamethylene biguanide (PHMB), sodium perborate or other agents known to those skilled in the art. Such preservatives are typically employed at a level of from 0.001 to 5.0% w/v. Unit dose compositions of the present invention will be sterile, but typically unpreserved. Such compositions, therefore, generally will not contain preservatives. The ophthalmic compositions of the present invention may also be provided preservative free and packaged in unit dose form.

In certain aspects of the invention, the pharmaceutical composition is substantially stable with respect to its chemical or physical attributes over a predetermined period of time. The measurable attributes may include, but are not limited to, percentage of active ingredient, percentage of impurities, pH, or visual attributes, such as color and the presence of particulates. In other aspects of the invention, the pharmaceutical composition is substantially stable following storage for about 4, 8, or 12 weeks at 25° C. In still other aspects of the invention, the pharmaceutical composition is substantially stable following storage for about 4, 8, or 12 weeks at 40° C. The methods for measuring the percentage of TRPA1 active ingredient and the percentage of impurities are well-known to a person skilled in the art.

In certain aspects, the invention provides an ocular formulation as described herein, wherein the compound of Formula I is a member selected from the group consisting of 1-(4-fluoro-3,5-dimethylphenyl)-8-{1-[4-fluoro-3-(trifluoromethyl)phenyl]-5-(pyridin-3-yl)-1H-pyrazole-3-carbonyl}-1,3,8-triazaspiro[4.5]decan-4-one; 6,7-difluoro-1-(1-{1-[4-fluoro-3-(trifluoromethyl)phenyl]-5-(pyridin-3-yl)-1H-pyrazole-3-carbonyl}piperidin-4-yl)-2,3-dihydro-1H-1,3-benzodiazol-2-one; 1′-{1-[4-fluoro-3-(trifluoromethyl)phenyl]-5-(pyridin-3-yl)-1H-pyrazole-3-carbonyl}-6-(trifluoromethyl)-1,2-dihydrospiro[3,1-benzoxazine-4,4′-piperidine]-2-one; 1′-{1-[4-fluoro-3-(trifluoromethyl)phenyl]-5-(pyridin-3-yl)-1H-pyrazole-3-carbonyl}-6-(trifluoromethyl)-1,2-dihydrospiro[3,1-benzoxazine-4,4′-piperidine]-2-one; 1′-{1-[4-fluoro-3-(trifluoromethyl)phenyl]-5-(pyridin-3-yl)-1H-pyrazole-3-carbonyl}-6-(trifluoromethyl)-1,2-dihydrospiro[3,1-benzoxazine-4,4′-piperidine]-2-one; 1′-{1-[4-fluoro-3-(trifluoromethyl)phenyl]-5-(pyridin-3-yl)-1H-pyrazole-3-carbonyl}-6-(trifluoromethyl)-1,2-dihydrospiro[3,1-benzoxazine-4,4′-piperidine]-2-one; 1′-{1-[4-fluoro-3-(trifluoromethyl)phenyl]-5-(pyridin-3-yl)-1H-pyrazole-3-carbonyl}-6-(trifluoromethyl)-1,2-dihydrospiro[3,1-benzoxazine-4,4′-piperidine]-2-one; 2-fluoro-4-{3-[1-(4-fluoro-3,5-dimethylphenyl)-4-oxo-1,3,8-triazaspiro[4.5]decane-8-carbonyl]-5-(pyridin-3-yl)-1H-pyrazol-1-yl}benzonitrile; 8-{1-[4-fluoro-3-(trifluoromethyl)phenyl]-5-(pyridin-3-yl)-1H-pyrazole-3-carbonyl}-1-(4-fluorophenyl)-1,3,8-triazaspiro[4.5]decan-4-one; 3-(1-{1-[4-fluoro-3-(trifluoromethyl)phenyl]-5-(pyridin-3-yl)-1H-pyrazole-3-carbonyl}piperidin-4-yl)-2-oxo-2,3-dihydro-1H-1,3-benzodiazole-5-carbonitrile; 6,7-difluoro-1-(1-{1-[4-iodo-3-(trifluoromethyl)phenyl]-5-(pyridin-3-yl)-1H-pyrazole-3-carbonyl}piperidin-4-yl)-2,3-dihydro-1H-1,3-benzodiazol-2-one; 6,7-difluoro-1-(1-{1-[4-iodo-3-(trifluoromethyl)phenyl]-5-(pyridin-3-yl)-1H-pyrazole-3-carbonyl}piperidin-4-yl)-2,3-dihydro-1H-1,3-benzodiazol-2-one; 8-{1-[4-fluoro-3-(trifluoromethyl)phenyl]-5-(pyridin-3-yl)-1H-pyrazole-3-carbonyl}-3′-(4-fluorophenyl)-8-azaspiro[bicyclo[3.2.1]octane-3,4′-imidazolidine]-5′-one; 6,7-difluoro-1-(1-{1-[4-fluoro-3-(trifluoromethyl)phenyl]-1H,4H,5H-pyrido[2,3-g]indazole-3-carbonyl}piperidin-4-yl)-2,3-dihydro-1H-1,3-benzodiazol-2-one; 5,6,7-trifluoro-1-(1-{1-[4-fluoro-3-(trifluoromethyl)phenyl]-5-(pyridin-3-yl)-1H-pyrazole-3-carbonyl}piperidin-4-yl)-2,3-dihydro-1H-1,3-benzodiazole-2-thione; 6,7-difluoro-1-(1-{1-[4-fluoro-3-methyl-5-(trifluoromethyl)phenyl]-5-(pyridin-3-yl)-1H-pyrazole-3-carbonyl}piperidin-4-yl)-2,3-dihydro-1H-1,3-benzodiazol-2-one; 6-fluoro-2-oxo-1-{1-[5-(pyridin-3-yl)-1-(3,4,5-trifluorophenyl)-1H-pyrazole-3-carbonyl]piperidin-4-yl}-2,3-dihydro-1H-1,3-benzodiazole-5-carbonitrile; 1-{1-[5-(6-chloropyridin-3-yl)-1-[3-fluoro-4-(trifluoromethyl)phenyl]-1H-pyrazole-3-carbonyl]piperidin-4-yl}-6,7-difluoro-2,3-dihydro-1H-1,3-benzodiazol-2-one; 6,7-difluoro-1′-{1-[4-fluoro-3-(trifluoromethyl)phenyl]-5-(pyridin-3-yl)-1H-pyrazole-3-carbonyl}-1,2-dihydrospiro[3,1-benzoxazine-4,4′-piperidine]-2-one; 6,7-difluoro-1-{1-[1-(4-iodophenyl)-5-(pyridin-3-yl)-1H-pyrazole-3-carbonyl]piperidin-4-yl}-2,3-dihydro-1H-1,3-benzodiazol-2-one; 1-(4-fluorophenyl)-8-[1-(4-iodophenyl)-5-(pyridin-3-yl)-1H-pyrazole-3-carbonyl]-1,3,8-triazaspiro[4.5]decan-4-one; 6,7-difluoro-1-(1-{1-[4-fluoro-3-(trifluoromethyl)phenyl]-1H,4H,5H-pyrido[3,2-g]indazole-3-carbonyl}piperidin-4-yl)-2,3-dihydro-1H-1,3-benzodiazol-2-one; 1-{1-[5-(2-chloropyridin-3-yl)-1-[3-fluoro-4-(trifluoromethyl)phenyl]-1H-pyrazole-3-carbonyl]piperidin-4-yl}-6,7-difluoro-2,3-dihydro-1H-1,3-benzodiazol-2-one; 6,7-difluoro-1-{8-[5-(pyridin-3-yl)-1-(3,4,5-trifluorophenyl)-1H-pyrazole-3-carbonyl]-8-azabicyclo[3.2.1]octan-3-yl}-2,3-dihydro-1H-1,3-benzodiazol-2-one; 1-(4-fluorophenyl)-8-{1-[4-iodo-3-(trifluoromethyl)phenyl]-5-(pyridin-3-yl)-1H-pyrazole-3-carbonyl}-1,3,8-triazaspiro[4.5]decan-4-one; 5,6,7-trifluoro-1-(1-{1-[4-fluoro-3-(trifluoromethyl)phenyl]-5-(pyridin-3-yl)-1H-pyrazole-3-carbonyl}piperidin-4-yl)-2,3-dihydro-1H-1,3-benzodiazol-2-one; 8-[5-(6-chloropyridin-3-yl)-1-[3-fluoro-4-(trifluoromethyl)phenyl]-1H-pyrazole-3-carbonyl]-1-(4-fluorophenyl)-1,3,8-triazaspiro[4.5]decan-4-one; 6,7-difluoro-1-(1-{1-[4-fluoro-3-(trifluoromethyl)phenyl]-5-(pyridin-3-yl)-1H-pyrazole-3-carbonyl}pyridin-4-yl)-2,3-dihydro-1H-1,3-benzodiazole-2-thione; 1-(1-{1-[3,4-bis(trifluoromethyl)phenyl]-5-(pyridin-3-yl)-1H-pyrazole-3-carbonyl}piperidin-4-yl)-6,7-difluoro-2,3-dihydro-1H-1,3-benzodiazol-2-one; 1-{1-[4-bromo-5-(pyridin-3-yl)-1-(3,4,5-trifluorophenyl)-1H-pyrazole-3-carbonyl]piperidin-4-yl}-6,7-difluoro-2,3-dihydro-1H-1,3-benzodiazol-2-one; (8aS)-7-{1-[3-fluoro-4-(trifluoromethyl)phenyl]-5-(pyridin-3-yl)-1H-pyrazole-3-carbonyl}-hexahydro-1H-[1,3]oxazolo[3,4-a]piperazin-3-one; 2-fluoro-5-{3-[1-(4-fluoro-3,5-dimethylphenyl)-4-oxo-1,3,8-triazaspiro[4.5]decane-8-carbonyl]-5-(pyridin-3-yl)-1H-pyrazol-1-yl}benzonitrile; 5,6-difluoro-1′-{1-[4-fluoro-3-(trifluoromethyl)phenyl]-5-(pyridin-3-yl)-1H-pyrazole-3-carbonyl}-1,2-dihydrospiro[3,1-benzoxazine-4,4′-piperidine]-2-one; 8-{1-[4-fluoro-3-(trifluoromethyl)phenyl]-5-(pyrazin-2-yl)-1H-pyrazole-3-carbonyl}-1-(4-fluorophenyl)-1,3,8-triazaspiro[4.5]decan-4-one; 6,7-difluoro-1-(1-{1-[4-fluoro-3-(trifluoromethyl)phenyl]-5-(pyrazin-2-yl)-1H-pyrazole-3-carbonyl}piperidin-4-yl)-2,3-dihydro-1H-1,3-benzodiazol-2-one; 8-{1-[4-fluoro-3-(trifluoromethyl)phenyl]-1H,4H,5H-pyrido[2,3-g]indazole-3-carbonyl}-1-1-(4-fluorophenyl)-1,3,8-triazaspiro[4.5]decan-4-one; 1-(3,5-dichloro-4-fluorophenyl)-8-{1-[4-fluoro-3-(trifluoromethyl)phenyl]-5-(pyridin-3-yl)-1H-pyrazole-3-carbonyl}-1,3,8-triazaspiro[4.5]decan-4-one; 8-{1-[3-fluoro-4-(trifluoromethyl)phenyl]-5-(pyridin-3-yl)-1H-pyrazole-3-carbonyl}-3′-(4-fluorophenyl)-8-azaspiro[bicyclo[3.2.1]octane-3,4′-imidazolidine]-5′-one; 6-fluoro-1-(1-{1-[3-fluoro-4-(trifluoromethyl)phenyl]-5-(pyridin-3-yl)-1H-pyrazole-3-carbonyl}piperidin-4-yl)-7-(trifluoromethyl)-2,3-dihydro-1H-1,3-benzodiazol-2-one; 8-{1-[4-chloro-3-(trifluoromethyl)phenyl]-5-(pyridin-3-yl)-1H-pyrazole-3-carbonyl}-1-(4-fluorophenyl)-1,3,8-triazaspiro[4.5]decan-4-one; 5-{3-[4-(6,7-difluoro-2-oxo-2,3-dihydro-1H-1,3-benzodiazol-1-yl)piperidine-1-carbonyl]-5-(pyridin-3-yl)-1H-pyrazol-1-yl}-2-fluorobenzonitrile; 4-{3-[1-(3,4-difluorophenyl)-4-oxo-1,3,8-triazaspiro[4.5]decane-8-carbonyl]-5-(pyridin-3-yl)-1H-pyrazol-1-yl}-2-fluorobenzonitrile; 1-(4-fluoro-3,5-dimethylphenyl)-8-{1-[3-fluoro-4-(trifluoromethyl)phenyl]-5-(pyridin-3-yl)-1H-pyrazole-3-carbonyl}-1,3,8-triazaspiro[4.5]decan-4-one; 4-{3-[4-(6,7-difluoro-2-oxo-2,3-dihydro-1H-1,3-benzodiazol-1-yl)piperidine-1-carbonyl]-5-(pyridin-3-yl)-1H-pyrazol-1-yl}-2-(trifluoromethyl)benzonitrile; 5-fluoro-3-(1-{1-[3-fluoro-4-(trifluoromethyl)phenyl]-5-(pyridin-3-yl)-1H-pyrazole-3-carbonyl}piperidin-4-yl)-2-oxo-2,3-dihydro-1H-1,3-benzodiazole-4-carbonitrile; 6,7-difluoro-1-(1-{1-[2-methyl-4-(trifluoromethoxy)phenyl]-5-(pyridin-3-yl)-1H-pyrazole-3-carbonyl}piperidin-4-yl)-2,3-dihydro-1H-1,3-benzodiazol-2-one; 1-(1-{1-[2,5-difluoro-4-(trifluoromethyl)phenyl]-5-(pyridin-3-yl)-1H-pyrazole-3-carbonyl}piperidin-4-yl)-6,7-difluoro-2,3-dihydro-1H-1,3-benzodiazol-2-one; 1-(1-{1-[3-fluoro-4-(trifluoromethyl)phenyl]-5-(pyridin-3-yl)-1H-pyrazole-3-carbonyl}piperidin-4-yl)-2-oxo-2,3-dihydro-1H-1,3-benzodiazole-5-carbonitrile; 1-(1-{1-[4-chloro-3-(trifluoromethyl)phenyl]-5-(pyridin-3-yl)-1H-pyrazole-3-carbonyl}piperidin-4-yl)-6,7-difluoro-2,3-dihydro-1H-1,3-benzodiazol-2-one; 6-fluoro-1-(1-{1-[3-fluoro-4-(trifluoromethyl)phenyl]-5-(pyridin-3-yl)-1H-pyrazole-3-carbonyl}piperidin-4-yl)-2-oxo-2,3-dihydro-1H-1,3-benzodiazole-5-carbonitrile; 1-(4-fluoro-3,5-dimethylphenyl)-8-[5-(pyridin-3-yl)-1-(3,4,5-trifluorophenyl)-1H-pyrazole-3-carbonyl]-1,3,8-triazaspiro[4.5]decan-4-one; 6-fluoro-1′-{1-[3-fluoro-4-(trifluoromethyl)phenyl]-5-(pyridin-3-yl)-1H-pyrazole-3-carbonyl}-1,2-dihydrospiro[3,1-benzoxazine-4,4′-piperidine]-2-one; 4-{1-[4-(6,7-difluoro-2-oxo-2,3-dihydro-1H-1,3-benzodiazol-1-yl)piperidine-1-carbonyl]-5-(pyridin-3-yl)-1H-pyrazol-1-yl}-2-fluorobenzonitrile; 6,7-difluoro-1-(1-{1-[3-fluoro-4-(trifluoromethyl)phenyl]-5-(4-methylpyridin-3-yl)-1H-pyrazole-3-carbonyl}piperidin-4-yl)-2,3-dihydro-1H-1,3-benzodiazol-2-one; 5-fluoro-2-oxo-3-{1-[5-(pyridin-3-yl)-1-(3,4,5-trifluorophenyl)-1H-pyrazole-3-carbonyl]piperidin-4-yl}-2,3-dihydro-1H-1,3-benzodiazole-4-carbonitrile; 1-(1-{1-[2,3-difluoro-4-(trifluoromethyl)phenyl]-5-(pyridin-3-yl)-1H-pyrazole-3-carbonyl}piperidin-4-yl)-6,7-difluoro-2,3-dihydro-1H-1,3-benzodiazol-2-one; 2-fluoro-4-(3-{[3′-(4-fluorophenyl)-5′-oxo-8-azaspiro[bicyclo[3.2.1]octane-3,4′-imidazolidine]-8-yl]carbonyl}-5-(pyridin-3-yl)-1H-pyrazol-1-yl)benzonitrile; 8-{1-[4-fluoro-3-(trifluoromethyl)phenyl]-1H,4H,5H-pyrido[3,2-g]indazole-3-carbonyl}-1-(4-fluorophenyl)-1,3,8-triazaspiro[4.5]decan-4-one; 6-fluoro-1′-{1-[4-fluoro-3-(trifluoromethyl)phenyl]-5-(pyridin-3-yl)-1H-pyrazole-3-carbonyl}-1,2-dihydrospiro[3,1-benzoxazine-4,4′-piperidine]-2-one; 6,7-difluoro-1-(1-{1-[3-fluoro-4-(trifluoromethyl)phenyl]-5-(5-fluoropyridin-3-yl)-1H-pyrazole-3-carbonyl}piperidin-4-yl)-2,3-dihydro-1H-1,3-benzodiazol-2-one; 6,7-difluoro-1-(8-{1-[4-fluoro-3-(trifluoromethyl)phenyl]-5-(pyridin-3-yl)-1H-pyrazole-3-carbonyl}-8-azabicyclo[3.2.1]octan-3-yl)-2,3-dihydro-1H-1,3-benzodiazol-2-one; 6,7-difluoro-1-(1-{1-[3-fluoro-4-(trifluoromethyl)phenyl]-5-(pyridazin-4-yl)-1H-pyrazole-3-carbonyl}piperidin-4-yl)-2,3-dihydro-1H-1,3-benzodiazol-2-one; 6-chloro-1′-{1-[4-fluoro-3-(trifluoromethyl)phenyl]-5-(pyridin-3-yl)-1H-pyrazole-3-carbonyl}-1,2-dihydrospiro[3,1-benzoxazine-4,4′-piperidine]-2-one; 6,7-difluoro-1-{1-[5-(pyridazin-4-yl)-1-(3,4,5-trifluorophenyl)-1H-pyrazole-3-carbonyl]piperidin-4-yl}-2,3-dihydro-1H-1,3-benzodiazol-2-one; 6,7-difluoro-1-(1-{1-[4-fluoro-3-(trifluoromethyl)phenyl]-5-(pyridin-3-yl)-1H-pyrazole-3-carbonyl}-2-methylpiperidin-4-yl)-2,3-dihydro-1H-1,3-benzodiazol-2-one; 7-fluoro-1′-{1-[4-fluoro-3-(trifluoromethyl)phenyl]-5-(pyridin-3-yl)-1H-pyrazole-3-carbonyl}-1,2-dihydrospiro[3,1-benzoxazine-4,4′-piperidine]-2-one; 1-(1-{1-[2,4-difluoro-3-(trifluoromethyl)phenyl]-5-(pyridin-3-yl)-1H-pyrazole-3-carbonyl}piperidin-4-yl)-6,7-difluoro-2,3-dihydro-1H-1,3-benzodiazol-2-one; 8-{1-[2,4-difluoro-3-(trifluoromethyl)phenyl]-5-(pyridin-3-yl)-1H-pyrazole-3-carbonyl}-1-(4-fluorophenyl)-1,3,8-triazaspiro[4.5]decan-4-one; 1-{1-[5-(1-benzyl-1H-imidazol-4-yl)-1-[4-fluoro-3-(trifluoromethyl)phenyl]-1H-pyrazole-3-carbonyl]piperidin-4-yl}-6,7-difluoro-2,3-dihydro-1H-1,3-benzodiazol-2-one; and 8-[5-(1-benzyl-1H-imidazol-4-yl)-1-[4-fluoro-3-(trifluoromethyl)phenyl]-1H-pyrazole-3-carbonyl]-1-(4-fluorophenyl)-1,3,8-triazaspiro[4.5]decan-4-one.

### III. Methods

In certain aspects, the invention provides a method for treating symptoms of dry eye, wherein the method comprises: administering to an eye of a mammal the ocular formulation as described in the aspects or embodiments herein.

In certain aspects, the invention provides a method for treating ocular pain or ocular inflammation, wherein the method comprises:

administering to an eye of a mammal the ocular formulation as described in the aspects or embodiments herein.

In certain other aspects, TRPA1 antagonists useful in the methods of the invention include, but are not limited to, fused azabicyclic, heterocyclic, and amide compounds as described, for example, in U.S. Patent Application Publication No. US 2011/0009430 A1. Other TRPA1 antagonists include, for example, those described in U.S. Pat. No. 9,260,430; International Patent Publication Nos. WO 2009/089083, 2009/147079, 2009/002933, 2009/118596, 2009/144548, 2010/004390, 2010/1388792007/073505, and 2010/039289; U.S. Pat. Appl. Publ. Nos. 2011/0009379 and 2010/0249154; and U.S. Pat. No. 7,671,061.

In certain preferred aspects, the invention provides a method as described herein, wherein the ocular formulation is topically administered. In certain aspects, the ocular formulation may be administered by intracameral, intravitreal, or periocular injection.

In certain aspects, the invention provides a method as described herein, wherein the ocular formulation is topically administered one to four times during a day. In certain aspects, the ocular formulation is topically administered once a day.

In certain aspects, the invention provides a method of treating dry eye as described herein, wherein the dry eye is associated with refractive surgery. In certain aspects, the refractive surgery is photorefractive keratectomy (PRK) surgery, laser epithelial keratomileusis (LASEK) refractive eye surgery, or laser-assisted in situ keratomileusis (LASIK) refractive eye surgery.

In certain aspects, the invention provides a method of treating ocular inflammation as described herein, wherein the ocular inflammation is caused by allergic, viral, or bacterial conjunctivitis; iritis; keratitis; injury from a chemical, radiation, or thermal burn; penetration of a foreign body; blepharitis; or scleritis.

In certain aspects, the invention provides a method of treating ocular pain as described herein, wherein the ocular pain is associated with refractive surgery. In certain aspects, the refractive surgery is photorefractive keratectomy (PRK) surgery, laser epithelial keratomileusis (LASEK) refractive eye surgery, or laser-assisted in situ keratomileusis (LASIK) refractive eye surgery.

In certain aspects, the invention provides a method for treating pain or inflammation associated with eye surgery, wherein the method comprises: pre-dosing a patient for a period up to 48 hours before the eye surgery with the ocular formulation as described herein, wherein the formulation is applied to an eye of the patient from one to three times daily; or applying the formulation post-surgery for about 14 days or until the pain or inflammation has been alleviated, wherein the formulation is applied to the eye of the patient from one to three times daily.

In certain aspects, the invention provides a method for treating pain or inflammation associated with eye surgery, wherein the method further comprises administering the ocular formulation to the eye of the patient periprocedurally, before, during or after eye surgery.

In certain preferred aspects, the invention provides a method for treating pain or inflammation associated with eye surgery, wherein the method comprises both pre-dosing the patient and applying the formulation post-surgery.

In certain aspects, the invention provides a method for treating pain or inflammation associated with eye surgery, wherein the eye surgery is cataract surgery.

In certain aspects, the invention provides a method for treating pain or inflammation associated with eye surgery, wherein the pain or inflammation occurs intraoperatively or during the postoperative eye surgery period. In certain aspects, the pain or inflammation is caused by postoperative eye surgery.

In certain aspects, the invention provides a method for treating pain or inflammation associated with eye surgery, wherein the pain is associated with refractive surgery. In certain aspects, the refractive surgery is photorefractive keratectomy (PRK) surgery, laser epithelial keratomileusis (LASEK) refractive eye surgery, or laser-assisted in situ keratomileusis (LASIK) refractive eye surgery.

In certain preferred aspects, TRPA1 antagonists are administered to a patient to prevent or ameliorate ocular pain associated with various stimuli. For example, the TRPA1 antagonists and compositions of the present invention may be used in treating pain arising from allergens, inflammation, trauma, dry eye, and/or foreign body sensation, such as from contact lenses and surgery. The compounds of the present invention may be used for the treatment of pain following ocular surgery, such as PRK surgery. With such treatment, the TRPA1 antagonists can be individually dosed, or in combination with other pharmaceutical agents such as by methods disclosed in U.S. Pat. No. 5,401,510 and PCT/US14/72291, the entire contents of which are incorporated herein by reference. The compounds will be utilized in a concentration or dose effective to prevent or ameliorate ocular pain and/or inflammation.

In certain aspects, a composition comprising one or more of the specified TRPA1 antagonists and a pharmaceutically acceptable carrier for topical ophthalmic administration or implantation into the conjunctival sac or injection into the anterior chamber of the eye is administered to a mammal in need thereof. The compositions are formulated in accordance with methods known in the art for the particular route of administration desired.

The pharmaceutical compositions administered according to the present invention comprise a pharmaceutically effective amount of one or more of the specified TRPA1 antagonists. As used herein, a “pharmaceutically effective amount” refers to that amount of one or more TRPA1 antagonists that prevents or alleviates ocular pain and/or is sufficient to reduce or eliminate symptoms of dry eye. Preferably, compositions are intended to be administered topically to the eye in the form of eye drops or eye ointments, wherein the total amount of TRPA1 antagonist will be about 0.001 to 5.0% (w/v). Preferably, the amount of TRPA1 antagonist is about 0.01 to about 2.0% (w/v) (e.g., about 0.01, 0.02, 0.03, 0.04, 0.05, 0.06, 0.07, 0.08, 0.09, 0.01, 0.011, 0.012, 0.013, 0.014, 0.015, 0.02, 0.025, 0.03, 0.035, 0.04, 0.045, 0.05, 0.06, 0.07, 0.08, 0.09, 0.10, 0.11, 0.12, 0.13, 0.14, 0.15, 0.16, 0.17, 0.18, 0.19, 0.2, 0.25, 0.3, 0.35, 0.4, 0.45, 0.5, 0.55, 0.6, 0.65, 0.7, 0.75, 0.8, 0.9, 1.0, 1.1, 1.2, 1.3, 1.4, 1.5, 1.6, 1.7, 1.8, 1.9, or 2.0%). Most preferably, the amount of the TRPA1 antagonist is about 0.05-1.0% (w/v) (e.g., about 0.05, 0.06, 0.07, 0.08, 0.09, 0.01, 0.011, 0.012, 0.013, 0.014, 0.015, 0.02, 0.021, 0.022, 0.023, 0.024, 0.025, 0.026, 0.027, 0.028, 0.029, 0.03, 0.033, 0.035, 0.038, 0.04, 0.045, 0.05, 0.06, 0.07, 0.08, 0.09, 0.10, 0.11, 0.12, 0.13, 0.14, 0.15, 0.16, 0.17, 0.18, 0.19, 0.2, 0.25, 0.3, 0.35, 0.4, 0.45, 0.5, 0.55, 0.6, 0.65, 0.7, 0.75, 0.8, 0.85, 0.9, 0.95, or 1.0%).

The preferred compositions of the present invention are intended for administration to a human patient suffering from ocular pain, inflammation, or symptoms of dry eye. Preferably, such compositions will be administered topically. The compositions may also be administered to the anterior segment of the eye periprocedurally in irrigation solutions. In general, the doses used for the above described purposes will vary, but will be an effective amount to reduce or eliminate ocular pain and/or eliminate or improve dry eye disease symptoms. Generally, 1-2 drops of such compositions will be administered one or more times per day. For example, the composition can be administered 2 to 3 times a day or as directed by an eye care provider. The volume of the drops may vary, but typically will be between 15-100 microliters.

In certain aspects, the invention provides a method for manufacture of a medicament as described in any of the aspects or embodiments herein for use in any of the methods of treatment described herein. In certain aspects, the invention provides a use of a fomulation as described in any of the aspects or embodiments herein for use in any of the methods described herein (e.g., treating dry eye disease).

### EXAMPLES

The following examples, including the experiments conducted and results achieved are provided for illustrative purposes only and are not to be construed as limiting the invention (e.g., the formulations are provided as representative formulations).

### Example 1: Formulations

A representative eye drop formulation is provided below.

| Ingredient | Amount (% w/v) |
|---|---|
| TRPA1 antagonist | 0.001-5.0 |
| Boric Acid | 0.25 |
| Sodium Chloride | 0.75 |
| Disodium Edetate | 0.01 |
| Polyquatemium-1 | 0.001 |
| NaOH/HCl | q.s., pH = 7.4 |
| Purified Water | q.s. 100% |

The above composition is prepared by the following method. The batch quantities of boric acid, sodium chloride, disodium edetate, and polyquaternium-1 are weighed and dissolved by stirring in 90% of the batch quantity of purified water or water for injection. The pH is adjusted to 7.4.±0.0.1 with NaOH and/or HCl. The batch quantity of the TRPA1 antagonist as a stock solution is measured and added. Purified water is added to q.s. to 100%. The mixture is stirred for five minutes to homogenize and then filtered through a sterilizing filter membrane into a sterile container.

### Example 2: Formulations Comprising Hydroxypropylmethylcellulose (HPMC)

The solutions below are suitable for both multi and single dose aqueous ophthalmic formulations for instillation into the eye and are composed of the drug substance (e.g., the TRPA1 antagonist) and a vehicle. The vehicle is made up of a buffered aqueous solution of viscosity enhancing agents, tonicity adjusting reagent with and without a preservative agent, e.g., polyquaternium-1 (PQ1 or Polyquad).

TABLE 2
Formulations Comprising HPMC

| | TRPA1 | | | 0.067 M | |
|---|---|---|---|---|---|
| Formulation | Antagonist | HPMC | PQ1 | NaH₂PO₄/ | NaCl |
| ID | % (w/v) | % (w/v) | % (w/v) | Na₂HPO4 Buffer | % (w/v) |
| 1 | 0.05 | 0.10 | 0.001 | pH 6.8-7.5 | 0.75 |
| 2 | 0.05 | 0.30 | 0.001 | pH 6.8-7.5 | 0.75 |
| 3 | 0.05 | 0.50 | 0.001 | pH 6.8-7.5 | 0.75 |
| 4 | 0.05 | 1.00 | 0.001 | pH 6.8-7.5 | 0.75 |
| 5 | 0.025 | 0.10 | 0.001 | pH 6.8-7.5 | 0.75 |
| 6 | 0.025 | 0.30 | 0.001 | pH 6.8-7.5 | 0.75 |
| 7 | 0.025 | 0.50 | 0.001 | pH 6.8-7.5 | 0.75 |
| 8 | 0.025 | 1.00 | 0.001 | pH 6.8-7.5 | 0.75 |
| 9 | 0.010 | 0.10 | 0.001 | pH 6.8-7.5 | 0.75 |
| 10 | 0.010 | 0.30 | 0.001 | pH 6.8-7.5 | 0.75 |
| 11 | 0.010 | 0.50 | 0.001 | pH 6.8-7.5 | 0.75 |
| 12 | 0.010 | 1.00 | 0.001 | pH 6.8-7.5 | 0.75 |
| 13 | 0.05 | 0.10 | 0.000 | pH 6.8-7.5 | 0.75 |
| 14 | 0.05 | 0.30 | 0.000 | pH 6.8-7.5 | 0.75 |
| 15 | 0.05 | 0.50 | 0.000 | pH 6.8-7.5 | 0.75 |

In certain embodiments, HPMC, also referred to as hypromellose, is dissolved in warm water (60-80° C.) by adding it at a rate of about 0.1 g/min with stirring until a final viscosity of between 35-60 cps is achieved and allowed to cool. The target concentration of HPMC in the drug product is between 0.1% -1% (w/v). A measured amount of the HPMC solution is added to the buffered solution to make the vehicle. The drug substance and, optionally, the preservative are dissolved in the vehicle and the tonicity of the final solution adjusted with 0.75% (w/v) sodium chloride solution. The target osmolality is between 280-310 mOSm/kg. The pH of the solution may be adjusted to the specified range of pH 6.8-7.4 using 1 N NaOH. The TRPA1 antagonist drug concentration in these formulations range between 0.01-0.05% (w/v), equivalent to 0.2 mM-1.0 mM.

### Example 3: TRPA1 Formulations Comprising Carboxymethylcellulose Sodium (CMC)

In certain embodiments, carboxymethylcellulose sodium salt (CMC) is dissolved in warm water (60 to 80° C.) by adding it at a rate of about 0.1 g/min with stirring until a final viscosity of 35-60 cps is achieved and allowed to cool. In certain aspects, the target concentration of CMC in the drug product is between 0.1% -1%. A measured amount of the CMC solution is added to the buffered solution to make the vehicle. The drug substance and the preservative are dissolved in the vehicle and the tonicity of the final solution adjusted with 0.75% (w/v) sodium chloride solution. Target osmolality is between 280-310 mOSm/kg. The pH of the solution may be adjusted to the specified range of pH 6.8-7.4 with 1 N NaOH.

TABLE 3
Formulations Comprising CMC

| | TRPA1 | | | 0.067 M | |
|---|---|---|---|---|---|
| Formulation | Antagonist | CMC | BAK | NaH₂PO₄/ | NaCl |
| ID | (% w/v) | (% w/v) | (% w/v) | Na₂HPO4 Buffer | (% w/v) |
| 1 | 0.05 | 0.10 | 0.01 | pH 6.8-7.5 | 0.75 |
| 2 | 0.05 | 0.30 | 0.01 | pH 6.8-7.5 | 0.75 |
| 3 | 0.05 | 0.50 | 0.01 | pH 6.8-7.5 | 0.75 |
| 4 | 0.05 | 1.00 | 0.01 | pH 6.8-7.5 | 0.75 |
| 5 | 0.025 | 0.10 | 0.01 | pH 6.8-7.5 | 0.75 |
| 6 | 0.025 | 0.30 | 0.01 | pH 6.8-7.5 | 0.75 |
| 7 | 0.025 | 0.50 | 0.01 | pH 6.8-7.5 | 0.75 |
| 8 | 0.025 | 1.00 | 0.01 | pH 6.8-7.5 | 0.75 |
| 9 | 0.010 | 0.10 | 0.01 | pH 6.8-7.5 | 0.75 |
| 10 | 0.010 | 0.30 | 0.01 | pH 6.8-7.5 | 0.75 |
| 11 | 0.010 | 0.50 | 0.01 | pH 6.8-7.5 | 0.75 |
| 12 | 0.010 | 1.00 | 0.01 | pH 6.8-7.5 | 0.75 |
| 13 | 0.05 | 0.10 | 0.00 | pH 6.8-7.5 | 0.75 |
| 14 | 0.05 | 0.30 | 0.00 | pH 6.8-7.5 | 0.75 |
| 15 | 0.05 | 0.50 | 0.00 | pH 6.8-7.5 | 0.75 |

### Example 4: TRPA1 Formulations Comprising Polyethylene Glycol (PEG) 400 and Propylene Glycol

In certain aspects, the vehicle for the TRPA1 drug substance is composed of a buffered solution of PEG 400 and propylene glycol. The drug substance and the preservative are dissolved in the vehicle and the tonicity of the final solution adjusted with 0.75% (w/v) sodium chloride solution. Target osmolality is between 280-310 mOsm/kg. The pH of the solution is checked and if necessary adjusted to the specified range of pH 6.8-7.4. In certain aspects, the concentration of PEG 400 in these solutions range between 0.2% -0.6% and propylene glycol content is between 0.2% and 0.5%.

TABLE 4
TRPA1 Formulations Comprising PEG 400 and Propylene Glycol

| | TRPA1 | | Propylene | | | |
|---|---|---|---|---|---|---|
| Formulation | Antagonist | PEG 400 | Glycol (PG) | PQ1 | 0.067 M NaH₂PO₄/ | NaCl |
| ID | (% w/v) | (% w/v) | (% w/v) | (% w/v) | Na₂HPO4 Buffer | (% w/v) |
| 1 | 0.05 | 0.2 | 0.20 | 0.001 | pH 6.8-7.5 | 0.75 |
| 2 | 0.05 | 0.30 | 0.30 | 0.001 | pH 6.8-7.5 | 0.75 |
| 3 | 0.05 | 0.40 | 0.40 | 0.001 | pH 6.8-7.5 | 0.75 |
| 4 | 0.05 | 0.60 | 0.50 | 0.001 | pH 6.8-7.5 | 0.75 |
| 5 | 0.025 | 0.20 | 0.20 | 0.001 | pH 6.8-7.5 | 0.75 |
| 6 | 0.025 | 0.30 | 0.30 | 0.001 | pH 6.8-7.5 | 0.75 |
| 7 | 0.025 | 0.40 | 0.40 | 0.001 | pH 6.8-7.5 | 0.75 |
| 8 | 0.025 | 0.60 | 0.50 | 0.001 | pH 6.8-7.5 | 0.75 |
| 9 | 0.010 | 0.20 | 0.20 | 0.001 | pH 6.8-7.5 | 0.75 |
| 10 | 0.010 | 0.30 | 0.30 | 0.001 | pH 6.8-7.5 | 0.75 |
| 11 | 0.010 | 0.40 | 0.40 | 0.001 | pH 6.8-7.5 | 0.75 |
| 12 | 0.010 | 0.60 | 0.50 | 0.001 | pH 6.8-7.5 | 0.75 |
| 13 | 0.05 | 0.20 | 0.20 | 0.000 | pH 6.8-7.5 | 0.75 |
| 14 | 0.05 | 0.30 | 0.30 | 0.000 | pH 6.8-7.5 | 0.75 |
| 15 | 0.05 | 0.50 | 0.50 | 0.000 | pH 6.8-7.5 | 0.75 |

### Example 5: TRPA1 Formulations Comprising Polyethylene Glycol (PEG) 400

In certain aspects, the vehicle for the TRPA1 drug substance is a buffered solution of PEG 400. The drug substance and the preservative are dissolved in the vehicle and the tonicity of the final solution adjusted with 0.75% (w/v) sodium chloride solution. The target osmolality is between 280-310 mOsm/kg. The pH of the solution is (if necessary) adjusted to the specified range of pH 6.8-7.4. In certain aspects, the concentration of PEG 400 in these formulations ranges between 0.2%-0.6%.

TABLE 5
Formulations Comprising PEG 400

| | TRPA1 | | | 0.067 M | |
|---|---|---|---|---|---|
| Formula- | Antagonist | PEG 400 | BAK | NaH₂PO₄/ | NaCl |
| tion ID | (% w/v) | (% w/v) | (% w/v) | Na₂HPO4 Buffer | (% w/v) |
| 1 | 0.05 | 0.2 | 0.01 | pH 6.8-7.5 | 0.75 |
| 2 | 0.05 | 0.30 | 0.01 | pH 6.8-7.5 | 0.75 |
| 3 | 0.05 | 0.40 | 0.01 | pH 6.8-7.5 | 0.75 |
| 4 | 0.05 | 0.60 | 0.01 | pH 6.8-7.5 | 0.75 |
| 5 | 0.025 | 0.20 | 0.01 | pH 6.8-7.5 | 0.75 |
| 6 | 0.025 | 0.30 | 0.01 | pH 6.8-7.5 | 0.75 |
| 7 | 0.025 | 0.40 | 0.01 | pH 6.8-7.5 | 0.75 |
| 8 | 0.025 | 0.60 | 0.01 | pH 6.8-7.5 | 0.75 |
| 9 | 0.010 | 0.20 | 0.01 | pH 6.8-7.5 | 0.75 |
| 10 | 0.010 | 0.30 | 0.01 | pH 6.8-7.5 | 0.75 |
| 11 | 0.010 | 0.40 | 0.01 | pH 6.8-7.5 | 0.75 |
| 12 | 0.010 | 0.60 | 0.01 | pH 6.8-7.5 | 0.75 |
| 13 | 0.05 | 0.20 | 0.00 | pH 6.8-7.5 | 0.75 |
| 14 | 0.05 | 0.30 | 0.00 | pH 6.8-7.5 | 0.75 |
| 15 | 0.05 | 0.50 | 0.00 | pH 6.8-7.5 | 0.75 |

In certain aspects, the vehicle for the TRPA1 drug substance is a buffered solution of PEG 400. The drug substance and the preservative are dissolved in the vehicle and the tonicity of the final solution adjusted with 0.75% (w/v) sodium chloride solution. The target osmolality is between 280-310 mOsm/kg. The pH of the solution is (if necessary) adjusted to the specified range of pH 6.8-7.4. In certain aspects, the concentration of PEG 400 in these formulations ranges between 0.2%-0.6%.

### Example 6: TRPA1 Formulations Comprising PEG 300

In certain aspects, the vehicle for the TRPA1 drug substance in these solutions is composed of buffered solution of PEG 300. The specified amount of drug substance and the preservative are dissolved in the vehicle and the tonicity of the final solution adjusted with 0.75% (w/v) sodium chloride solution. Target osmolality is between 280-310 mOsm/kg. The pH of the solution is checked and if necessary adjusted to the specified range of pH 6.8-7.4. In certain aspects, the concentration of PEG300 in these formulations ranges between 0.2%-0.6%.

TABLE 6
Formulations Comprising PEG 300

| | TRPA1 | | | 0.067 M | |
|---|---|---|---|---|---|
| Formula- | Antagonist | PEG 300 | BAK | NaH₂PO₄/ | NaCl |
| tion ID | (% w/v) | (% w/v) | (% w/v) | Na₂HPO4 Buffer | (% w/v) |
| 1 | 0.05 | 0.2 | 0.01 | pH 6.8-7.5 | 0.75 |
| 2 | 0.05 | 0.30 | 0.01 | pH 6.8-7.5 | 0.75 |
| 3 | 0.05 | 0.40 | 0.01 | pH 6.8-7.5 | 0.75 |
| 4 | 0.05 | 0.60 | 0.01 | pH 6.8-7.5 | 0.75 |
| 5 | 0.025 | 0.20 | 0.01 | pH 6.8-7.5 | 0.75 |
| 6 | 0.025 | 0.30 | 0.01 | pH 6.8-7.5 | 0.75 |
| 7 | 0.025 | 0.40 | 0.01 | pH 6.8-7.5 | 0.75 |
| 8 | 0.025 | 0.60 | 0.01 | pH 6.8-7.5 | 0.75 |
| 9 | 0.010 | 0.20 | 0.01 | pH 6.8-7.5 | 0.75 |
| 10 | 0.010 | 0.30 | 0.01 | pH 6.8-7.5 | 0.75 |
| 11 | 0.010 | 0.40 | 0.01 | pH 6.8-7.5 | 0.75 |
| 12 | 0.010 | 0.60 | 0.01 | pH 6.8-7.5 | 0.75 |
| 13 | 0.05 | 0.20 | 0.00 | pH 6.8-7.5 | 0.75 |
| 14 | 0.05 | 0.30 | 0.00 | pH 6.8-7.5 | 0.75 |
| 15 | 0.05 | 0.50 | 0.00 | pH 6.8-7.5 | 0.75 |

### Example 7: TRPA1 Emulsion Formulations

In certain aspects, the vehicle for the TRPA1 modulator (e.g., an antagonist) in these solutions is an emulsion formulation. In certain aspects, the emulsion formulation comprises a second active compound.

The hydrophobic component preferably is present in the emulsion composition in an amount greater than about 0.625% by weight more preferably in amount up to about 1.0% by weight or about 1.5% by weight or more of the composition. The hydrophobic component preferably comprises one or more oily substances and may be comprised of castor oil, olive oil or any combination thereof or any animal or vegetable oil, mineral oils and without limitation any higher fatty acid glycerides.

The emulsion composition may include emulsion stabilizing agent including crosslinked polyacrylates such as carbomers. Preferably, the emulsion stabilizing agent may be from the acrylate/C10-30 alkyl acrylate cross-polymers class, or higher molecular weight co-polymers of acrylic acid and such classes of related compounds.

The emulsion formulation may include useful polyanionic components to provide viscosity and stabilize the emulsion. Such a polyelectrolyte/emulsion stabilizing agent may be present in an amount in a range about 0.01% by weight or less to about 1% by weight or more preferably about 0.02% by weight to about 0.5% by weight of the composition.

Suitable tonicity agent would be employed in the present composition and would include such tonicity components which are non-ionic in order to not interfere with the other components of the emulsion formulation described herein. Useful tonicity agents may include, but are not limited to glycerin, mannitol, sorbitol, and the like. The present composition may include tonicity agents, preferably in the range of about 10-20% of isotonicity.

Ophthalmic viscosity enhancing agents may be included in the emulsion formulation in effective amounts, preferably in the range of about 0.01%, about 0.5%, about 1.0% by weight. In certain aspects, the viscosity enhancing agent is included in the emulsion formulation in an amount ranging from 0.02% to about 0.5% by weight.

The pH of the composition can be adjusted in a conventional manner using sodium hydroxide and hydrochloric acid to a pH range of 6 to about 10, preferably about 7.0 to about 8.0 and still more preferably about 7.2 to about 7.6.

A buffer system may be incorporated in the present composition. When a buffer is needed to maintain suitable pH, representative buffer systems (without limitation) would include phosphates, citrates, acetates, borates, and the like.

The present composition may include preservative at an effective level. Such preservatives may include (without limitation) benzalkonium chloride, methyl and ethyl parabens, hexetidine, and phenyl mercuric salts.

The oil-in-water emulsion system employed in this formulations can be sterilized separately before final composition or sterile filtered, or terminal sterilized by heat such as autoclave steam sterilization.

TABLE 7
Exemplary compositions of combination TRPA1 antagonist and
cyclosporine emulsion formulations.

| | Composition (% w/v) | | | | | | |
|---|---|---|---|---|---|---|---|
| Component | 1 | 2 | 3 | 4 | 5 | 6 | 7 |
| Cyclosporin A | 0.1 | 0.1 | 0.01 | 0.01 | 0.1 | 0.05 | 0.05 |
| TRPA1 antagonist | 0.1 | 0.01 | 0.01 | 0.1 | 0.05 | 0.2 | 0.05 |
| Castor Oil | 1.25 | 1.25 | 1.25 | 1.25 | 1.25 | 1.25 | 1.25 |
| Polysorbate 80 | 1.0 | 1.0 | 1.0 | 1.0 | 1.0 | 1.0 | 1.0 |
| acrylate/C10-30 alkyl | 0.05 | 0.05 | 0.05 | 0.05 | 0.05 | 0.05 | 0.05 |
| acrylate (Pemulen ®) | | | | | | | |
| Glycerol | 2.20 | 2.20 | 2.20 | 2.20 | 2.20 | 2.20 | 2.20 |
| Sodium Hydroxide | qs | qs | qs | qs | qs | qs | qs |
| Purified Water | qs | qs | qs | qs | qs | qs | qs |
| pH | 7.2-7.7 | 7.0-7.6 | 7.2-7.6 | 7.2-7.6 | 7.2-7.6 | 7.2-7.6 | 7.2-7.6 |

qs = quantum satis (the amount that is needed)

### Example 8: Effect of TRPA1 Antagonists on Ocular Pain

The effects of transient receptor potential receptor subfamily A, member 1 (TRPA1) antagonists on ocular pain in rats is tested using a formalin-induced blink response assay. Sprague-Dawley rats are treated with topical ocular administration of 20 μL of vehicle, and the composition to be tested is applied to one eye only. After the appropriate pretreatment time of about 5 minutes, 5 μL of 0.1% formalin is applied topically to the eye. Each rat is placed in a clear plastic box, and the number of blinks is counted for 1 minute immediately following the formalin challenge. Additional time periods and intervals are included to establish time-dependent changes for each test group. Test groups include vehicle control and multiple concentrations or doses of a TRPA1 antagonist (e.g., 0.05, 0.10, or 0.20%w/v).

A decrease in the results of the blink response assay indicates inhibition of the formalin-induced blink response in a dose-dependent fashion, showing greater inhibition at the highest TRPA1 antagonist concentration to be tested in an ocular formulation of the present invention.

### Example 9: Measuring TRPA1 Antagonist Ocular Topical Analgesic Activities

Corneal analgesic effects of TRPA1 antagonists are examined by analyzing suppression of blinks induced by mechanical touch. A Cochet-Bonnet Esthesiometer is used to determine corneal anesthetic activities of the TRPA1 antagonist in normal rats.

Male Sprague-Dawley rats (200-300 g) are divided into groups of 6 each, restrained in a DecapiCone rat restraint, and secured at the posterior with tape. A hole is cut into the cone to expose the right eye. Twenty-four hours before the experiment, the eyelashes and whiskers are trimmed with scissors. The right eye is dosed with 20 pl of drug or vehicle, and the timer is set for 5 minutes to allow the rat time to acclimate. The Cochet-Bonnet Esthesiometer fiber is set at 30 mm and is perpendicularly touched by a masked observer to the center of the cornea 10 times with a 3 second delay between counts. Blinks are counted with each touch of the fiber, and a total score out of 10 is recorded. If more than one blink occurs in response to a single touch, this event is counted as one blink response.

The topical anesthetic, 0.5% proparacaine (Alcaine), is used to inhibit the mechanical blink response by about 95%, providing an anesthetic drug reference for the study. The TRPA1 composition to be tested is not anticipated to completely inhibit the blink response, as its activity is expected to be analgesic (i.e., reduced pain sensation) rather than anesthetic (i.e., no sensation). Thus, the effects of a TRPA1 antagonist on ocular pain and corneal sensitivity can be measured and shown to produce significant topical analgesic activity, while not exhibiting topical anesthetic activity.

### Example 10: Efficacy Testing of TRPA1 Antagonists in Models of Dry Eye Disease

Validated experimental animal models that mimic dry eye disease (DED) or dry eye syndrome (DES) in the human eye are known and employed for preclinical testing in the field of ophthalmology. These preclinical animal models can be satisfactorily used to run a dry eye proof-of-concept study and to test and select therapeutic candidates. A variety of animal models in multiple species are available to mimic the different pathophysiologic mechanisms of DED.

### Mouse Lacrimal Gland Dry Eye Model

C57BL/6 J wild-type mice are obtained from Jackson Laboratory. Adult male mice are anesthetized with a xylazine (3 mg/g) and ketamine (15 mg/g) mixture and incisions of 5 mm are made in the skin between the eye and the ear, and both extraorbital lacrimal glands are gently isolated by forceps and removed. As the mouse has three pairs of lacrimal glands, removing only the extraorbital glands induces partially tear secretion deficiency, but the mice still have other tear sources. Mice in the sham group (control) receive the same procedure without gland removal. The skin is then sutured with 6-0 black monofilament nylon (Ethilon from Ethicon, Inc.). All mice receive antibiotics (100 μL Enroflox™, intramuscular daily) and topical analgesia (2% lidocaine gel) for 2 days in the postoperative period. Behavioral assays are conducted between 2 and 4 weeks after the surgery. Each study group of mice includes from 7-10 mice per group.

Tear volume is measured with phenol-red cotton threads as previously described by methods well-known in the art. The threads are held with forceps and applied to the lateral canthus for 30 s. Immediately afterwards, the wetting of the thread is read in mm under a dissection microscope. Corneal abrasion is assessed under cobalt blue light after application of 0.5 μl of 0.25% fluorescein sodium. Grades of abrasion are classified with a grading system that is based on area of corneal staining. Grouping is blinded to the observers. Results are grouped according to the treatments after analysis. Analysis can be conducted a multiple and various study timepoints. Corneal fluorescein staining is one endpoint for measurement. Additional endpoints, including hyperemia and ocular surface sensitivity as measured by a Cochet-Bonnet ethesiometer in the study.

Standard statistical analysis, such as using one-way analysis of variance followed by a Tukey-Kramer post-hoc test is used for comparison of vehicle and each TRPA1 antagonist dose groups. To compare various parameters in each time point, repeated measure analysis of variance followed by Bonferroni's post-hoc analysis is used. Dry eye animal efficacy models based on a controlled adverse environment or use of anti-cholinergic drugs

In a mouse or rat model, dry eye disease can be induced using 21-day systemic and continuous delivery of scopolamine through an osmotic pump implanted subcutaneously. In a mouse model, DED is caused by placing the mice in a controlled adverse environment. Mice are placed in a controlled environmental chamber with a relative humidity of less than 25% (e.g. 15%), an air flow of 15 liters per minute, and scopolamine, an anti-muscarinic cholinergic drug that minimizes tear production, is administered. Both rodent models using scopolamine effectively induce dry eye by causing a rapid decrease in tear production and an increase in corneal defects. Study endpoints and statistical analysis are the same as described for the lacrimal gland removal model.

It should be understood that the foregoing disclosure emphasizes certain specific embodiments of the invention and that all modifications or alternatives equivalent thereto are within the spirit and scope of the invention as set forth in the appended claims. All patents and other publications disclosed herein are incorporated by reference in their entirety.

What is claimed is:

1. An ocular formulation, wherein the ocular formulation comprises
(i) a pharmaceutically acceptable carrier comprising
an aqueous buffer,
a viscosity enhancing agent, and
an opthalmologically acceptable inorganic salt or tonicity agent; and
(ii) a pharmaceutically effective amount of a TRPA1 antagonist;
wherein the TRPA1 antagonist is a compound of Formula I: 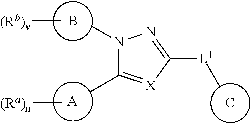
or a pharmaceutically acceptable salt thereof;
wherein:
A is a cyclic group of Formula Ia: 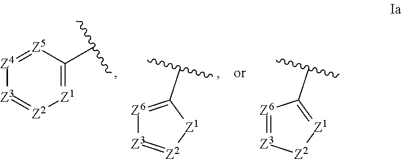
wherein Z¹, Z², Z³, Z⁴, Z⁵, and Z⁶ are each a member selected from the group consisting of N, CH, CR^{a}, and NR^{c}; or, alternatively for Z¹ or Z⁶, the member Z¹ or Z⁶ and X, together with atoms in the rings to which they are attached, form an additional fused, five- to eight-membered cycloalkyl or heterocyclyl ring with from 0 to 4 R^{z} substituents;
with the proviso that at least one member selected from the group consisting of Z², Z³, Z⁴, and Z⁶ is N;
each R^{z} is a member independently selected from the group consisting of halo, C₁-C₃ alkyl, C₁-C₃ haloalkyl, and C₁-C₃ alkoxy; or, alternatively, two R^{z} substituents, together with the carbon atom to which they are attached, join to form an oxo, spirocycloalkyl, or spiroheterocyclyl group;
B is a cyclic group of Formula Ib: 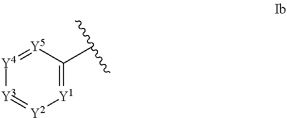
wherein Y¹, Y², Y³, Y⁴, and Y⁵ are each a member independently selected from the group consisting of N, CH, and CR^{b}; or, alternatively, the members —Y²═Y³— or —Y⁴═Y⁵— are combined into a single member selected from the group consisting of NR^{c}, O, and S;
each IV and R^{b} is a member independently selected from the group consisting of cyano, carboxyl, C₁-C₄ alkyl, C₁-C₄ hydroxyalkyl, C₃-C₈ cycloalkyl, C₁-C₄ alkoxy, C₁-C₄ haloalkyl, C₁-C₄ haloalkoxy, halo, Cₒ-C₆ amino, C₁-C₆ amido, C₁-C₄ alkyloxycarbonyl, C₁-C₆ alkylsulfonyl, and hydroxyl; or, alternatively, two adjacent R^{a} or R^{b}, together with the atoms in groups A or B to which they are attached, form an additional fused aryl, heteroaryl, cycloalkyl, or heterocyclyl ring with from 0 to 4 R^{z} substituents;
each R^{c} is a member independently selected from the group consisting of hydrogen, C₁-C₄ alkyl, C₁-C₄ haloalkyl, C₃-C₆ cycloalkyl, (C₃-C₆ cycloalkyl)C₁-C₃alkyl, and C₁-C₇ acyl;
each u is an integer independently selected from 0 to 4;
v is an integer from 0 to 5;
X is N or CR^{d}; or, alternatively, X is CR^{d}, wherein X and the member Z¹, together with atoms in the rings in which they are included, form the additional fused, five- to eight-membered cycloalkyl or heterocyclyl ring with from 0 to 4 R^{z} substituents;
each R^{d} is a member independently selected from the group consisting of hydrogen, halo, cyano, C₁-C₄ alkyl, C₁-C₄ haloalkyl, C₁-C₄ alkoxy, C₁-C₄ haloalkoxy, C₃-C₆ cycloalkyl, and C₃-C₆ cycloalkoxy;
each L¹, L², and L³, if present, is a member independently selected from the group consisting of C═O, C═S, and C═NR^{c};
C is a cyclic group of Formula Ic: 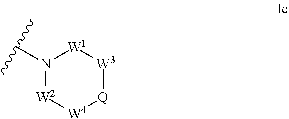
wherein Q is a member selected from the group consisting of C(R^{e})(D), N(E), F, and G; or, alternatively, the members -W³-Q- or -W⁴-Q- join to form a member H; and
wherein W¹, W², W³, and W⁴ are each an independently selected C(R^{f})₂; or, alternatively, the members -W³-Q- or -W⁴-Q- join to form a member H;
R^{c} is a member selected from the group consisting of hydrogen, C₁-C₃ alkyl, and C₁-C₃ fluoroalkyl; or, alternatively, R^{e} and an R^{f} substituent of W¹, W², W³, or W⁴ join to form a —(C(R^{z})₂)ₜ— bridge, wherein t is an integer selected from 2 or 3;
each R^{f} is a member independently selected from the group consisting of hydrogen, C₁-C₃ alkyl, C₁-C₃ fluoroalkyl, and halo; or, alternatively, two adjacent R^{f}, together with the atoms in group C to which they are attached, form an additional aryl, heteroaryl, cycloalkyl, or heterocyclyl fused ring with from 0 to 4 R^{z} substituents; or, alternatively, two geminal R^{f}, together with the atom in group C to which they are attached, form a spirocycloalkyl or spiroheterocyclyl ring with from 0 to 4 R^{z} substituents; or, alternatively, two axial R^{f} substituents of a pair of Wⁿ selected from the group consisting of (W¹ and W²), (W² and W³), and (W³ and W⁴) join to form a —(C(R^{z})₂)ₜ— bridge; or, alternatively, R^{e} and an R^{f} substituent of W¹, W², W³, or W⁴ join to form a —(C(R^{z})₂)ₜ— bridge;
each t is an integer selected from 2 or 3;
D is a bicyclic group of Formula Id: 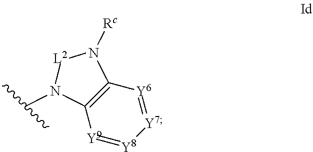
E is a bicyclic group of Formula Ie: 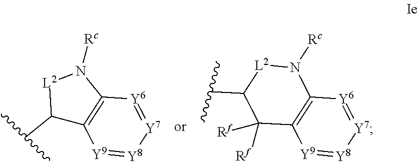
F is a spirocyclic group of Formula If: 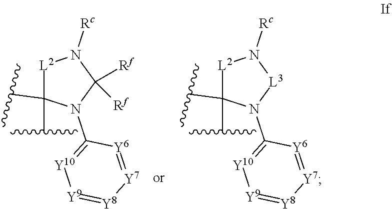
G is a bicyclic spirocyclic group of Formula Ig: 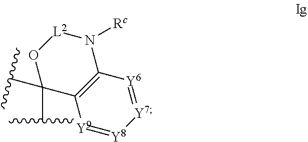
H is a fused group of Formula Ih: 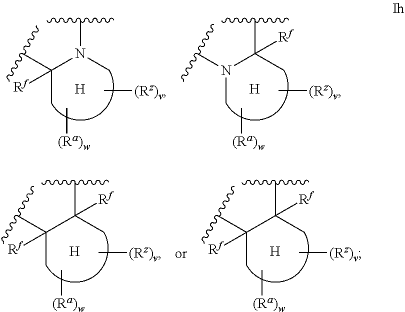
wherein the H ring is a fused, five- to eight-membered cycloalkyl or heterocyclyl ring;
wherein v is an integer from 0 to 4; and
wherein w is an integer from 0 to 2; and
Y⁶, Y⁷, Y⁸, Y⁹, and Y¹⁰, if present, are each a member independently selected from the group consisting of N, CH, and CR^{b}; or, alternatively for Y⁸ and Y⁹, the members —Y⁶═Y⁷— or —Y⁸═Y⁹— are combined into a single member selected from the group consisting of NR^{c}, O, and S.

2. The ocular formulation of claim 1, wherein the ocular formulation comprises
(i) a pharmaceutically acceptable carrier; and
(ii) a pharmaceutically effective amount of a TRPA1 antagonist;
wherein the TRPA1 antagonist is a compound of Formula I: 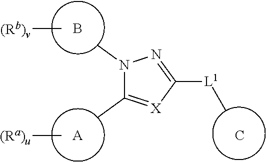
or a pharmaceutically acceptable salt thereof;
wherein:
A is a cyclic group of Formula la: 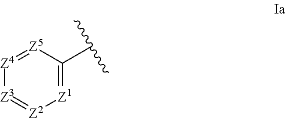
wherein Z¹, Z², Z³, Z⁴, and Z⁵ are each a member selected from the group consisting of N, CH, and CR^{a}; or, alternatively for Z¹, the member Z¹ and X, together with atoms in the rings to which they are attached, form an additional fused, five- to eight-membered cycloalkyl or heterocyclyl ring with from 0 to 4 R^{z} substituents;
with the proviso that at least one member selected from the group consisting of Z², Z³, and Z⁴ is N;
each R^{z} is a member independently selected from the group consisting of halo, C₁-C₃ alkyl, C₁-C₃ haloalkyl, and C₁-C₃ alkoxy; or, alternatively, two R^{z} substituents, together with the carbon atom to which they are attached, join to form an oxo, spirocycloalkyl, or spiroheterocyclyl group;
B is a cyclic group of Formula Ib: 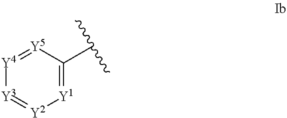
wherein Y¹, Y², Y³, Y⁴, and Y⁵ are each a member independently selected from the group consisting of N, CH, and CR^{b}; or, alternatively, the members —Y²═Y³— or —Y⁴═Y⁵— are combined into a single member selected from the group consisting of NH, NR^{c}, O, and S;
each R^{a} and R^{b} is a member independently selected from the group consisting of cyano, carboxyl, C₁-C₄ alkyl, C₁-C₄ hydroxyalkyl, C₃-C₈ cycloalkyl, C₁-C₄ alkoxy, C₁-C₄ haloalkyl, C₁-C₄ haloalkoxy, halo, C₀-C₆ amino, C₁-C₆ amido, C₁-C₄ alkyloxycarbonyl, C₁-C₆ alkylsulfonyl, and hydroxyl; or, alternatively, two adjacent R^{a} or R^{b}, together with the atoms in groups A or B to which they are attached, form an additional fused aryl, heteroaryl, cycloalkyl, or heterocyclyl ring with from 0 to 4 R^{z} substituents;
each R^{c} is a member independently selected from the group consisting of C₁-C₄ alkyl, C₁-C₄ haloalkyl, C₃-C₆ cycloalkyl, (C₃-C₆ cycloalkyl)C₁-C₃alkyl, and C₁-C₇ acyl;
each u is an integer independently selected from 0 to 4;
v is an integer from 0 to 5;
X is N or CR^{d}; or, alternatively, X is CR^{d}, wherein X and the member Z¹, together with atoms in the rings in which they are included, form the additional fused, five- to eight-membered cycloalkyl or heterocyclyl ring with from 0 to 4 R^{z} substituents;
each R^{d} is a member independently selected from the group consisting of hydrogen, halo, cyano, C₁-C₄ alkyl, C₁-C₄ haloalkyl, C₁-C₄ alkoxy, C₁-C₄ haloalkoxy, C₃-C₆ cycloalkyl, and C₃-C₆ cycloalkoxy;
each L¹, L², and L³, if present, is a member independently selected from the group consisting of C═O, C═S, C═NH, and C═NR^{c};
C is a cyclic group of Formula Ic: 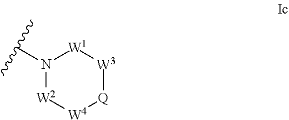
wherein Q is a member selected from the group consisting of C(R^{e})(D), N(E), F, and G; or, alternatively, the members —W³-Q- or —W⁴-Q- join to form a member H; and
wherein W¹, W², W³, and W⁴ are each an independently selected C(R^{f})₂; or, alternatively, the members —W³-Q- or —W⁴-Q- join to form a member H;
R^{e} is a member selected from the group consisting of hydrogen, C₁-C₃ alkyl, and C₁-C₃ fluoroalkyl; or, alternatively, R^{e} and an R^{f} substituent of W¹, W², W³, or W⁴ join to form a —(C(R^{z} )₂)ₜ— bridge, wherein t is an integer selected from 2 or 3;
each R^{f} is a member independently selected from the group consisting of hydrogen, C₁-C₃ alkyl, C₁-C₃ fluoroalkyl, and halo; or, alternatively, two adjacent R^{f}, together with the atoms in group C to which they are attached, form an additional aryl, heteroaryl, cycloalkyl, or heterocyclyl fused ring with from 0 to 4 R^{z} substituents; or, alternatively, two geminal R^{f}, together with the atom in group C to which they are attached, form a spirocycloalkyl or spiroheterocyclyl ring with from 0 to 4 R^{z} substituents; or, alternatively, two axial R^{f} substituents of a pair of Wₙ selected from the group consisting of (W¹ and W²), (W² and W³), and (W³ and W⁴) join to form a —(C(R^{z} )₂)ₜ— bridge; or, alternatively, R^{e} and an R^{f} substituent of W¹, W², W³, or W⁴ join to form a —(C(R^{z})₂)ₜ— bridge;
each t is an integer selected from 2 or 3;
D is a bicyclic group of Formula Id: 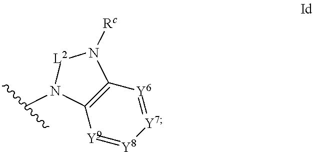
E is a bicyclic group of Formula Ie: 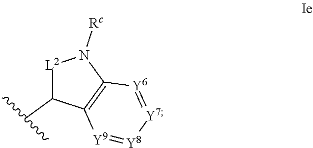
F is a spirocyclic group of Formula If: 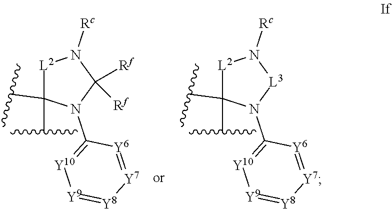
G is a bicyclic spirocyclic group of Formula Ig: 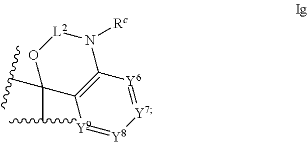
H is a fused group of Formula Ih: 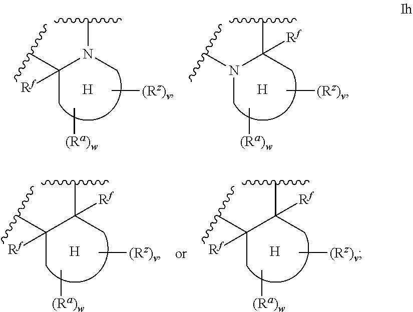
wherein the H ring is a fused, five- to eight-membered cycloalkyl or heterocyclyl ring;
wherein v is an integer from 0 to 4; and
wherein w is an integer from 0 to 2; and
Y⁶, Y⁷, Y⁸, Y⁹, and Y¹⁰ if present, are each a member independently selected from the group consisting of N, CH, and CR^{b}; or, alternatively for Y⁸ and Y⁹, the members —Y⁶═Y⁷— or —Y⁸═Y⁹— are combined into a single member selected from the group consisting of NH, NR^{c}, O, and S.

3. The ocular formulation of claim 2, wherein Q is a member selected from the group consisting of C(R^{e})(D) and F; and
wherein each L² is a member independently selected from the group consisting of C═O and C═S.

4. The ocular formulation of claim 3, wherein D is a member selected from the group consisting of:

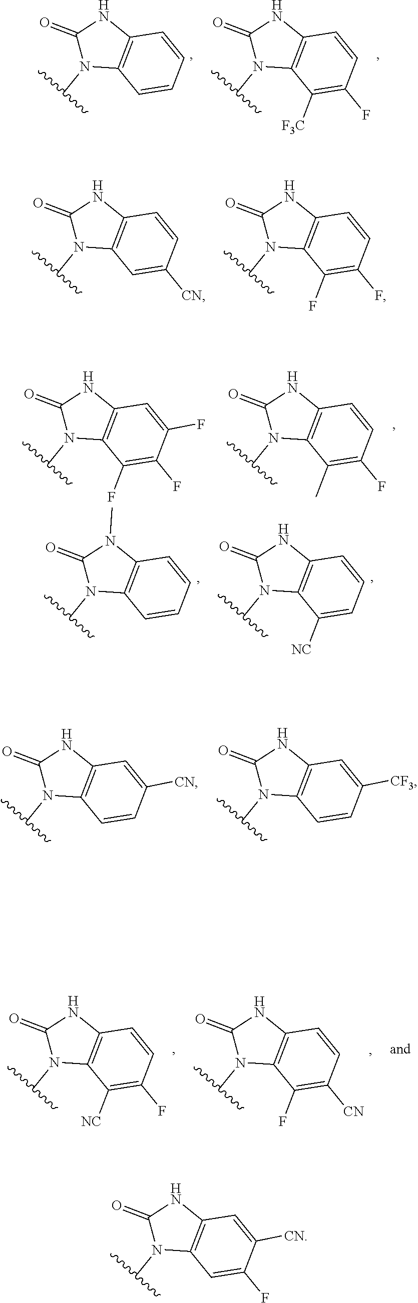

5. The ocular formulation of claim 3, wherein D is a member selected from the group consisting of:

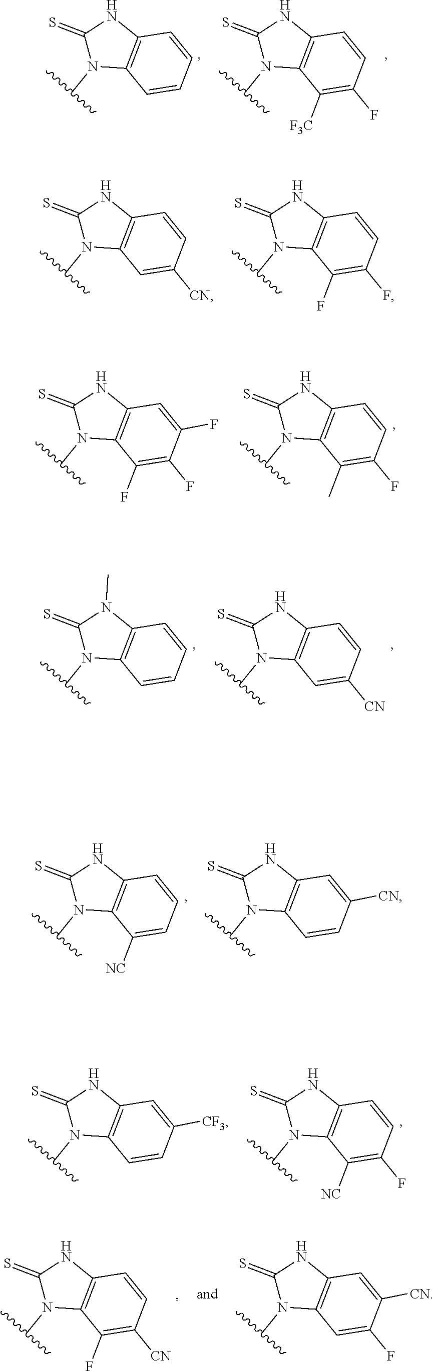

6. The ocular formulation of claim 2, wherein A is a member selected from the group consisting of: 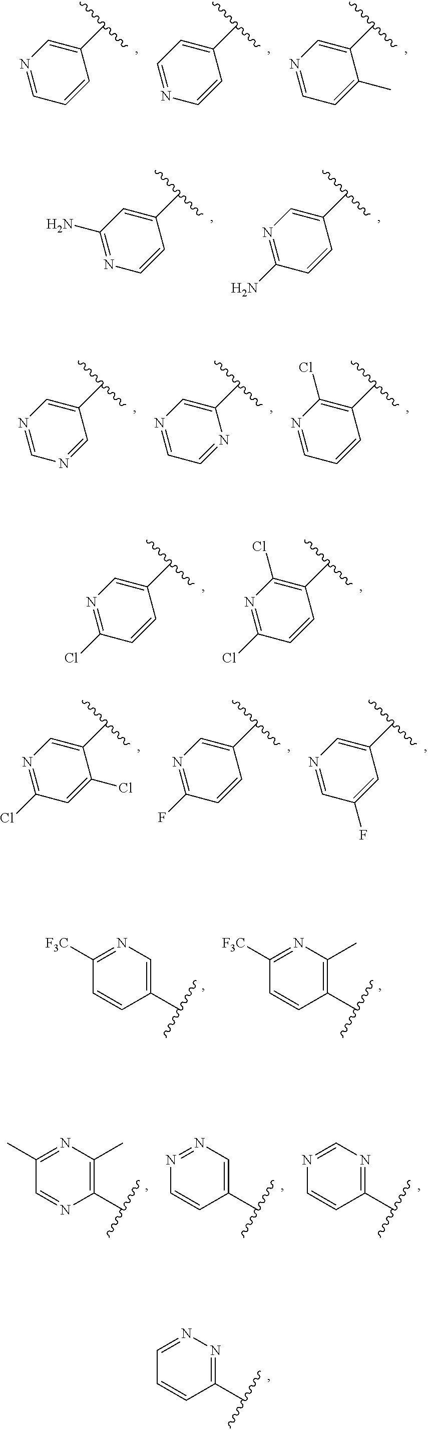
and a salt thereof.

7. The ocular formulation of any one of the preceding claims, wherein B is a member selected from the group consisting of:

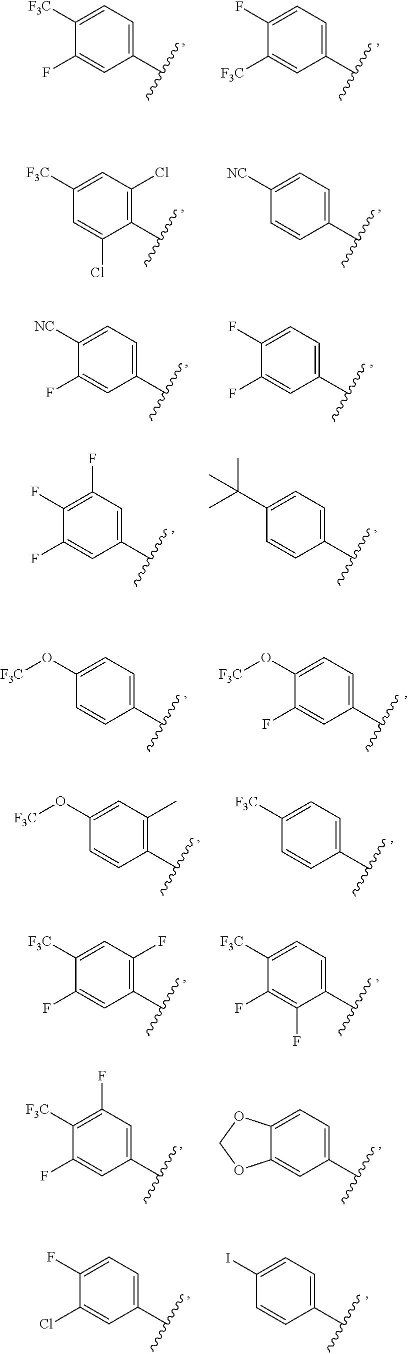

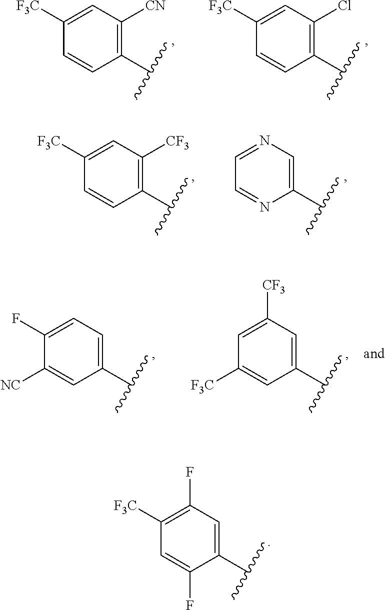

8. The ocular formulation of any one of the preceding claims, wherein the pharmaceutically effective amount of the TRPA1 antagonist is from 0.001% to 5.0% (w/v).

9. The ocular formulation of claim 8, wherein the pharmaceutically effective amount of the TRPA1 antagonist is from about 0.01% to 2.0% (w/v).

10. The ocular formulation of claim 9, wherein the pharmaceutically effective amount of the TRPA1 antagonist is from about 0.01% to 0.05% (w/v).

11. The ocular formulation of any one of the preceding claims, wherein the carrier comprises the tonicity agent.

12. The ocular formulation of any one of the preceding claims, wherein the aqueous buffer is a borate-boric acid system, a citrate-citric acid system, a phosphate-based system, an acetate-acetic acid system, or a combination thereof

13. The ocular formulation of any one of the preceding claims, wherein the ocular formulation has a pH of from about 6 to 10.

14. The ocular formulation of claim 13, wherein the ocular formulation has a pH of from about 6.3 to 8.0.

15. The ocular formulation of claim 14, wherein the pH is from about 6.8 to 7.5.

16. The ocular formulation of claim 14, wherein the pH is from about 7.0 to 8.0.

17. The ocular formulation of any one of the preceding claims, wherein the ocular formulation comprises a viscosity enhancing agent.

18. The ocular formulation of any one of the preceding claims, wherein the viscosity enhancing agent is selected from the group consisting of a polysaccharide, a dextran, a polyacrylate, a polyvinyl, and a combination thereof

19. The ocular formulation of claim 18, wherein the viscosity enhancing agent is selected from the group consisting of 0.1% to 1.5% (w/v) hydroxypropylmethylcellulose, 0.1% to 1.2% (w/v) hydroxyethylcellulose, 0.1% to 2.5% (w/v) methylcellulose, 0.1% to 1.6% (w/v) polyvinyl alcohol, 0.1% to 2.0% (w/v) polyvinyl pyrrolidine, 0.1% to 5.0% (w/v) polysorbate, 0.1% to 5.0% (w/v) polyethylene glycol, 0.1% to 2.5% (w/v) carboxymethylcellulose, 0.1% to 1.0% (w/v) propylene glycol, 0.1% to 1.0% hyaluronic acid, 0.1% to 2% (w/v) hyaluronate salt, and a combination thereof.

20. The ocular formulation of any one of the preceding claims, wherein the ocular formulation comprises the opthalmologically acceptable inorganic salt.

21. The ocular formulation of claim 20, wherein the opthalmologically acceptable inorganic salt is sodium chloride, potassium chloride, or a combination thereof.

22. The ocular formulation of any one of the preceding claims, wherein the ocular formulation comprises the tonicity agent, and wherein the tonicity agent is selected from the group consisting of dextrose, glycerin, mannitol, and a combination thereof.

23. The ocular formulation of any one of the preceding claims, wherein the ocular formulation has an osmolality of between about 170 mOSm/kg and 500 mOSm/kg.

24. The ocular formulation of claim 23, wherein the ocular formulation has an osmolality of between about 250 mOSm/kg and 350 mOSm/kg.

25. The ocular formulation of claim 24, wherein the ocular formulation has an osmolality of between about 280 mOSm/kg and 310 mOSm/kg.

26. The ocular formulation of any one of the preceding claims, wherein the ocular formulation comprises a non-ionic surfactant.

27. The ocular formulation of claim 26, wherein the non-ionic surfactant is from about 0.01% w/v to about 0.5% w/v.

28. The ocular formulation of claim 27, wherein the non-ionic surfactant is from about 0.02% w/v to about 0.3% w/v.

29. The ocular formulation of claim 26, wherein the non-ionic surfactant is an alkyl aryl polyether alcohol or a polyoxyethylene alkyl ether.

30. The ocular formulation of claim 29, wherein the alkyl aryl polyether alcohol is tyloxapol.

31. The ocular formulation of claim 26, wherein the non-ionic surfactant is selected from the group consisting of Brij 35, Brij 78, Brij 98, Brij 700, Pluronic F127, Polysorbate 20, Polysorbate 40, Polysorbate 60, Polysorbate 80, Solulan C-24, Span 20, Span, 40, Span 60, and Span 80.

32. The ocular formulation of any one of the preceding claims, wherein the formulation comprises a second drug that is selected from the group consisting of an antibiotic, a mydriatic, a local anesthetic, a non-steroidal anti-inflammatory drug, a steroidal anti-inflammatory drug, a drug for treatment of low tear production, an anti-histamine, and a combination thereof.

33. The ocular formulation of claim 32, wherein the second drug is cyclosporine A, azithromycin, or a combination thereof

34. The ocular formulation of any one of the preceding claims, wherein the ocular formulation is an emulsion.

35. The ocular formulation of claim 34, wherein the ocular formulation comprises an oil that is selected from the group consisting of an animal oil, a vegetable oil, a fatty acid glyceride, and a combination thereof

36. The ocular formulation of claim 35, wherein the oil is castor oil, olive oil, or a combination thereof.

37. The ocular formulation of claim 35, wherein the ocular formulation comprises from about 0.625% (w/v) to about 1.5% (w/v) of the oil.

38. The ocular formulation of claim 34, wherein the ocular formulation comprises a carbomer.

39. The ocular formulation of claim 8, wherein the ocular formulation has an pH of between about 6.0 and 10.0; and
wherein the ocular formulation has an osmolality of between about 170 mOSm/kg and 350 mOSm/kg.

40. The ocular formulation of any one of the preceding claims, wherein the ocular formulation comprises a preservative.

41. The ocular formulation of claim 40, wherein the preservative is selected from the group consisting of a quaternary ammonium compound, hexetidine, an alkyl mercury salt, a paraben, and a combination thereof

42. The ocular formulation of claim 41, wherein the preservative is benzalkonium chloride, methyl paraben, ethyl paraben, hexetidine, a phenyl mercuric salt, chlorobutanol, propylparaben, phenylethyl alcohol, edetate disodium, sorbic acid, polyquaternium-1, thiomersal, polyhexamethylene biguanide, sodium perborate, a SofZia buffer system, or a combination thereof

43. The ocular formulation of any one of the preceding claims, wherein the compound of Formula I is a member selected from the group consisting of 1-(4-fluoro-3,5-dimethylphenyl)-8-{1-[4-fluoro-3-(trifluoromethyl)phenyl]-5-(pyridin-3-yl)-1H-pyrazole-3-carbonyl}-1,3,8-triazaspiro[4.5]decan-4-one; 6,7-difluoro-1-(1-{1-[4-fluoro-3-(trifluoromethyl)phenyl]-5-(pyridin-3-yl)-1H-pyrazole-3-carbonyl}piperidin-4-yl)-2,3-dihydro-1H-1,3-benzodiazol-2-one; 1′-{1-[4-fluoro-3-(trifluoromethyl)phenyl]-5-(pyridin-3-yl)-1H-pyrazole-3-carbonyl}-6-(trifluoromethyl)-1,2-dihydrospiro[3,1-benzoxazine-4,4′-piperidine]-2-one; 1′-{1-[4-fluoro-3-(trifluoromethyl)phenyl]-5-(pyridin-3-yl)-1H-pyrazole-3-carbonyl}-6-(trifluoromethyl)-1,2-dihydrospiro[3,1-benzoxazine-4,4′-piperidine]-2-one; 1′-{1-[4-fluoro-3-(trifluoromethyl)phenyl]-5-(pyridin-3-yl)-1H-pyrazole-3-carbonyl}-6-(trifluoromethyl)-1,2-dihydrospiro[3,1-benzoxazine-4,4′-piperidine]-2-one; 1′-{1-[4-fluoro-3-(trifluoromethyl)phenyl]-5-(pyridin-3-yl)-1H-pyrazole-3-carbonyl}-6-(trifluoromethyl)-1,2-dihydrospiro[3,1-benzoxazine-4,4′-piperidine]-2-one; 1′-{1-[4-fluoro-3-(trifluoromethyl)phenyl]-5-(pyridin-3-yl)-1H-pyrazole-3-carbonyl}-6-(trifluoromethyl)-1,2-dihydrospiro[3,1-benzoxazine-4,4′-piperidine]-2-one; 2-fluoro-4-{3-[1-(4-fluoro-3,5-dimethylphenyl)-4-oxo-1,3,8-triazaspiro[4.5]decane-8-carbonyl]-5-(pyridin-3-yl)-1H-pyrazol-1-yl}benzonitrile; 8-{1-[4-fluoro-3-(trifluoromethyl)phenyl]-5-(pyridin-3-yl)-1H-pyrazole-3-carbonyl}-1-(4-fluorophenyl)-1,3,8-triazaspiro[4.5]decan-4-one; 3-(1-{1-[4-fluoro-3-(trifluoromethyl)phenyl]-5-(pyridin-3-yl)-1H-pyrazole-3-carbonyl}piperidin-4-yl)-2-oxo-2,3-dihydro-1H-1,3-benzodiazole-5-carbonitrile; 6,7-difluoro-1-(1-{1-[4-iodo-3-(trifluoromethyl)phenyl]-5-(pyridin-3-yl)-1H-pyrazole-3-carbonyl}piperidin-4-yl)-2,3-dihydro-1H-1,3-benzodiazol-2-one; 6,7-difluoro-1-(1-{1-[4-iodo-3-(trifluoromethyl)phenyl]-5-(pyridin-3-yl)-1H-pyrazole-3-carbonyl}piperidin-4-yl)-2,3-dihydro-1H-1,3-benzodiazol-2-one; 8-{1-[4-fluoro-3-(trifluoromethyl)phenyl]-5-(pyridin-3-yl)-1H-pyrazole-3-carbonyl}-3′-(4-fluorophenyl)-8-azaspiro[bicyclo[3.2.1]octane-3,4′-imidazolidine]-5′-one; 6,7-difluoro-1-(1-{1-[4-fluoro-3-(trifluoromethyl)phenyl]-1H,4H,5H-pyrido[2,3-g]indazole-3-carbonyl}piperidin-4-yl)-2,3-dihydro-1H-1,3-benzodiazol-2-one; 5,6,7-trifluoro-1-(1-{1-[4-fluoro-3-(trifluoromethyl)phenyl]-5-(pyridin-3-yl)-1H-pyrazole-3-carbonyl}piperidin-4-yl)-2,3-dihydro-1H-1,3-benzodiazole-2-thione; 6,7-difluoro-1-(1-{1-[4-fluoro-3-methyl-5-(trifluoromethyl)phenyl]-5-(pyridin-3-yl)-1H-pyrazole-3-carbonyl}piperidin-4-yl)-2,3-dihydro-1H-1,3-benzodiazol-2-one; 6-fluoro-2-oxo-1-{1-[5-(pyridin-3-yl)-1-(3,4,5-trifluorophenyl)-1H-pyrazole-3-carbonyl]piperidin-4-yl}-2,3-dihydro-1H-1,3-benzodiazole-5-carbonitrile; 1-{1-[5-(6-chloropyridin-3-yl)-1-[3-fluoro-4-(trifluoromethyl)phenyl]-1H-pyrazole-3-carbonyl]piperidin-4-yl}-6,7-difluoro-2,3-dihydro-1H-1,3-benzodiazol-2-one; 6,7-difluoro-1′-{1-[4-fluoro-3-(trifluoromethyl)phenyl]-5-(pyridin-3-yl)-1H-pyrazole-3-carbonyl}-1,2-dihydrospiro[3,1-benzoxazine-4,4′-piperidine]-2-one; 6,7-difluoro-1-{1-[1-(4-iodophenyl)-5-(pyridin-3-yl)-1H-pyrazole-3-carbonyl}piperidin-4-yl)-2,3-dihydro-1H-1,3-benzodiazol-2-one; 1-(4-fluorophenyl)-8-[1-(4-iodophenyl)-5-(pyridin-3-yl)-1H-pyrazole-3-carbonyl]-1,3,8-triazaspiro[4.5]decan-4-one; 6,7-difluoro-1-(1-{1-[4-fluoro-3-(trifluoromethyl)phenyl]-1H,4H,5H-pyrido[3,2-g]indazole-3-carbonyl}piperidin-4-yl)-2,3-dihydro-1H-1,3-benzodiazol-2-one; 1-{1-[5-(2-chloropyridin-3-yl)-1-[3-fluoro-4-(trifluoromethyl)phenyl]-1H-pyrazole-3-carbonyl]piperidin-4-yl}-6,7-difluoro-2,3-dihydro-1H-1,3-benzodiazol-2-one; 6,7-difluoro-1-{8-[5-(pyridin-3-yl)-1-(3,4,5-trifluorophenyl)-1H-pyrazole-3-carbonyl]-8-azabicyclo[3.2.1]octan-3-yl}-2,3-dihydro-1H-1,3-benzodiazol-2-one; 1-(4-fluorophenyl)-8-{1-[4-iodo-3-(trifluoromethyl)phenyl]-5-(pyridin-3-yl)-1H-pyrazole-3-carbonyl}-1,3,8-triazaspiro[4.5]decan-4-one; 5,6,7-trifluoro-1-(1-{1-[4-fluoro-3-(trifluoromethyl)phenyl]-5-(pyridin-3-yl)-1H-pyrazole-3-carbonyl}piperidin-4-yl)-2,3-dihydro-1H-1,3-benzodiazol-2-one; 8-[5-(6-chloropyridin-3-yl)-1-[3-fluoro-4-(trifluoromethyl)phenyl]-1H-pyrazole-3-carbonyl]-1-(4-fluorophenyl)-1,3,8-triazaspiro[4.5]decan-4-one; 6,7-difluoro-1-(1-{1-[4-fluoro-3-(trifluoromethyl)phenyl]-5-(pyridin-3-yl)-1H-pyrazole-3-carbonyl}pyridin-4-yl)-2,3-dihydro-1H-1,3-benzodiazole-2-thione; 1-(1-{1-[3,4-bis(trifluoromethyl)phenyl]-5-(pyridin-3-yl)-1H-pyrazole-3-carbonyl}piperidin-4-yl)-6,7-difluoro-2,3-dihydro-1H-1,3-benzodiazol-2-one; 1-{1-[4-bromo-5-(pyridin-3-yl)-1-(3,4,5-trifluorophenyl)-1H-pyrazole-3-carbonyl]piperidin-4-yl}-6,7-difluoro-2,3-dihydro-1H-1,3-benzodiazol-2-one; (8aS)-7-{1-[3-fluoro-4-(trifluoromethyl)phenyl]-5-(pyridin-3-yl)-1H-pyrazole-3-carbonyl}-hexahydro-1H-[1,3]oxazolo[3,4-a]piperazin-3-one; 2-fluoro-5-{3-[1-(4-fluoro-3,5-dimethylphenyl)-4-oxo-1,3,8-triazaspiro[4.5]decane-8-carbonyl]-5-(pyridin-3-yl)-1H-pyrazol-1-yl}benzonitrile; 5,6-difluoro-1′-{1-[4-fluoro-3-(trifluoromethyl)phenyl]-5-(pyridin-3-yl)-1H-pyrazole-3-carbonyl}-1,2-dihydrospiro[3,1-benzoxazine-4,4′-piperidine]-2-one; 8-{1-[4-fluoro-3-(trifluoromethyl)phenyl]-5-(pyrazin-2-yl)-1H-pyrazole-3-carbonyl}-1-(4-fluorophenyl)-1,3,8-triazaspiro[4.5]decan-4-one; 6,7-difluoro-1-(1-{1-[4-fluoro-3-(trifluoromethyl)phenyl]-5-(pyrazin-2-yl)-1H-pyrazole-3-carbonyl}piperidin-4-yl)-2,3-dihydro-1H-1,3-benzodiazol-2-one; 8-{1-[4-fluoro-3-(trifluoromethyl)phenyl]-1H,4H,5H-pyrido[2,3-g]indazole-3-carbonyl}-1-1-(4-fluorophenyl)-1,3,8-triazaspiro[4.5]decan-4-one; 1-(3,5-dichloro-4-fluorophenyl)-8-{1-[4-fluoro-3-(trifluoromethyl)phenyl]-5-(pyridin-3-yl)-1H-pyrazole-3-carbonyl}-1,3,8-triazaspiro[4.5]decan-4-one; 8-{1-[3-fluoro-4-(trifluoromethyl)phenyl]-5-(pyridin-3-yl)-1H-pyrazole-3-carbonyl}-3′-(4-fluorophenyl)-8-azaspiro[bicyclo[3.2.1]octane-3,4′-imidazolidine]-5′-one; 6-fluoro-1-(1-{1-[3-fluoro-4-(trifluoromethyl)phenyl]-5-(pyridin-3-yl)-1H-pyrazole-3-carbonyl}piperidin-4-yl)-7-(trifluoromethyl)-2,3-dihydro-1H-1,3-benzodiazol-2-one; 8-{1-[4-chloro-3-(trifluoromethyl)phenyl]-5-(pyridin-3-yl)-1H-pyrazole-3-carbonyl}-1-(4-fluorophenyl)-1,3,8-triazaspiro[4.5]decan-4-one; 5-{3-[4-(6,7-difluoro-2-oxo-2,3-dihydro-1H-1,3-benzodiazol-1-yl)piperidine-1-carbonyl]-5-(pyridin-3-yl)-1H-pyrazol-1-yl}-2-fluorobenzonitrile; 4-{3-[1-(3,4-difluorophenyl)-4-oxo-1,3,8-triazaspiro[4.5]decane-8-carbonyl]-5-(pyridin-3-yl)-1H-pyrazol-1-yl1-2-fluorobenzonitrile; 1-(4-fluoro-3,5-dimethylphenyl)-8-{1-[3-fluoro-4-(trifluoromethyl)phenyl]-5-(pyridin-3-yl)-1H-pyrazole-3-carbonyl}-1,3,8-triazaspiro[4.5]decan-4-one; 4-1344-(6,7-difluoro-2-oxo-2,3-dihydro-1H-1,3-benzodiazol-1-yl)piperidine-1-carbonyl]-5-(pyridin-3-yl)-1H-pyrazol-1-yl}-2-(trifluoromethyl)benzonitrile; 5-fluoro-3-(1-{1-[3-fluoro-4-(trifluoromethyl)phenyl]-5-(pyridin-3-yl)-1H-pyrazole-3-carbonyl}piperidin-4-yl)-2-oxo-2,3-dihydro-1H-1,3-benzodiazole-4-carbonitrile; 6,7-difluoro-1-(1-{1-[2-methyl-4-(trifluoromethoxy)phenyl]-5-(pyridin-3-yl)-1H-pyrazole-3-carbonyl}piperidin-4-yl)-2,3-dihydro-1H-1,3-benzodiazol-2-one; 1-(1-{1-[2,5-difluoro-4-(trifluoromethyl)phenyl]-5-(pyridin-3-yl)-1H-pyrazole-3-carbonyl}piperidin-4-yl)-6,7-difluoro-2,3-dihydro-1H-1,3-benzodiazol-2-one; 1-(1-{1-[3-fluoro-4-(trifluoromethyl)phenyl]-5-(pyridin-3-yl)-1H-pyrazole-3-carbonyl}piperidin-4-yl)-2-oxo-2,3-dihydro-1H-1,3-benzodiazole-5-carbonitrile; 1-(1-{1-[4-chloro-3-(trifluoromethyl)phenyl]-5-(pyridin-3-yl)-1H-pyrazole-3-carbonyl}piperidin-4-yl)-6,7-difluoro-2,3-dihydro-1H-1,3-benzodiazol-2-one; 6-fluoro-1-(1-{1-[3-fluoro-4-(trifluoromethyl)phenyl]-5-(pyridin-3-yl)-1H-pyrazole-3-carbonyl}piperidin-4-yl)-2-oxo-2,3-dihydro-1H-1,3-benzodiazole-5-carbonitrile; 1-(4-fluoro-3,5-dimethylphenyl)-8-[5-(pyridin-3-yl)-1-(3,4,5-trifluorophenyl)-1H-pyrazole-3-carbonyl]-1,3,8-triazaspiro[4.5]decan-4-one; 6-fluoro-1′-{1-[3-fluoro-4-(trifluoromethyl)phenyl]-5-(pyridin-3-yl)-1H-pyrazole-3-carbonyl}-1,2-dihydrospiro[3,1-benzoxazine-4,4′-piperidine]-2-one; 4-{1-[4-(6,7-difluoro-2-oxo-2,3-dihydro-1H-1,3-benzodiazol-1-yl)piperidine-1-carbonyl]-5-(pyridin-3-yl)-1H-pyrazol-1-yl}-2-fluorobenzonitrile; 6,7-difluoro-1-(1-{1-[3-fluoro-4-(trifluoromethyl)phenyl]-5-(4-methylpyridin-3-yl)-1H-pyrazole-3-carbonyl}piperidin-4-yl)-2,3-dihydro-1H-1,3-benzodiazol-2-one; 5-fluoro-2-oxo-3-{1-[5-(pyridin-3-yl)-1-(3,4,5-trifluorophenyl)-1H-pyrazole-3-carbonyl]piperidin-4-yl}-2,3-dihydro-1H-1,3-benzodiazole-4-carbonitrile; 1-(1-{1-[2,3-difluoro-4-(trifluoromethyl)phenyl]-5-(pyridin-3-yl)-1H-pyrazole-3-carbonyl}piperidin-4-yl)-6,7-difluoro-2,3-dihydro-1H-1,3-benzodiazol-2-one; 2-fluoro-4-(3-{[3′-(4-fluorophenyl)-5′-oxo-8-azaspiro[bicyclo[3.2.1]octane-3,4′-imidazolidine]-8-yl]carbonyl}-5-(pyridin-3-yl)-1H-pyrazol-1-yl)benzonitrile; 8-{1-[4-fluoro-3-(trifluoromethyl)phenyl]-1H,4H,5H-pyrido[3,2-g]indazole-3-carbonyl}-1-(4-fluorophenyl)-1,3,8-triazaspiro[4.5]decan-4-one; 6-fluoro-1′-{1-[4-fluoro-3-(trifluoromethyl)phenyl]-5-(pyridin-3-yl)-1H-pyrazole-3-carbonyl}-1,2-dihydrospiro[3,1-benzoxazine-4,4′-piperidine]-2-one; 6,7-difluoro-1-(1-{1-[3-fluoro-4-(trifluoromethyl)phenyl]-5-(5-fluoropyridin-3-yl)-1H-pyrazole-3-carbonyl}piperidin-4-yl)-2,3-dihydro-1H-1,3-benzodiazol-2-one; 6,7-difluoro-1-(8-{1-[4-fluoro-3-(trifluoromethyl)phenyl]-5-(pyridin-3-yl)-1H-pyrazole-3-carbonyl}-8-azabicyclo[3.2.1]octan-3-yl)-2,3-dihydro-1H-1,3-benzodiazol-2-one; 6,7-difluoro-1-(1-{1-[3-fluoro-4-(trifluoromethyl)phenyl]-5-(pyridazin-4-yl)-1H-pyrazole-3-carbonyl}piperidin-4-yl)-2,3-dihydro-1H-1,3-benzodiazol-2-one; 6-chloro-1′-{1-[4-fluoro-3-(trifluoromethyl)phenyl]-5-(pyridin-3-yl)-1H-pyrazole-3-carbonyl}-1,2-dihydrospiro[3,1-benzoxazine-4,4′-piperidine]-2-one; 6,7-difluoro-1-{1-[5-(pyridazin-4-yl)-1-(3,4,5-trifluorophenyl)-1H-pyrazole-3-carbonyl]piperidin-4-yl}-2,3-dihydro-1H-1,3-benzodiazol-2-one; 6,7-difluoro-1-(1-{1-[4-fluoro-3-(trifluoromethyl)phenyl]-5-(pyridin-3-yl)-1H-pyrazole-3-carbonyl}-2-methylpiperidin-4-yl)-2,3-dihydro-1H-1,3-benzodiazol-2-one; 7-fluoro-1′-{1-[4-fluoro-3-(trifluoromethyl)phenyl]-5-(pyridin-3-yl)-1H-pyrazole-3-carbonyl}-1,2-dihydrospiro[3,1-benzoxazine-4,4′-piperidine]-2-one; 1-(1-{1-[2,4-difluoro-3-(trifluoromethyl)phenyl]-5-(pyridin-3-yl)-1H-pyrazole-3-carbonyl}piperidin-4-yl)-6,7-difluoro-2,3-dihydro-1H-1,3-benzodiazol-2-one; 8-{1-[2,4-difluoro-3-(trifluoromethyl)phenyl]-5-(pyridin-3-yl)-1H-pyrazole-3-carbonyl}-1-(4-fluorophenyl)-1,3,8-triazaspiro[4.5]decan-4-one; 1-{1-[5-(1-benzyl-1H-imidazol-4-yl)-1-[4-fluoro-3-(trifluoromethyl)phenyl]-1H-pyrazole-3-carbonyl]piperidin-4-yl}-6,7-difluoro-2,3-dihydro-1H-1,3-benzodiazol-2-one; and 8-[5-(1-benzyl-1H-imidazol-4-yl)-1-[4-fluoro-3-(trifluoromethyl)phenyl]-1H-pyrazole-3-carbonyl]-1-(4-fluorophenyl)-1,3,8-triazaspiro[4.5]decan-4-one.

44. A method for treating symptoms of dry eye, wherein the method comprises:
administering to an eye of a mammal the ocular formulation of any one of claims 1 to 43.

45. The method of claim 44, wherein the ocular formulation is topically administered.

46. The method of claim 44 wherein the ocular formulation is topically administered one to four times during a day.

47. The method of claim 46 wherein the ocular formulation is topically administered once a day.

48. The method of claim 44, wherein the dry eye is associated with refractive surgery.

49. The method of claim 48, wherein the refractive surgery is photorefractive keratectomy (PRK) surgery, laser epithelial keratomileusis (LASEK) refractive eye surgery, or laser-assisted in situ keratomileusis (LASIK) refractive eye surgery.

50. A method for the treatment of ocular pain or ocular inflammation, wherein the method comprises:
administering to an eye of a mammal the ocular formulation of any one of claims 1 to 43.

51. The method of claim 50, wherein the ocular formulation is topically administered.

52. The method of claim 50, wherein the ocular formulation is topically administered one to four times during a day.

53. The method of claim 52, wherein the ocular formulation is topically administered once a day.

54. The method of claim 52, wherein the ocular inflammation is caused by allergic, viral, or bacterial conjunctivitis; iritis; keratitis; injury from a chemical, radiation, or thermal burn; penetration of a foreign body; blepharitis; or scleritis.

55. The method of claim 50, wherein the ocular pain is associated with refractive surgery.

56. The method of claim 55, wherein the refractive surgery is photorefractive keratectomy (PRK) surgery, laser epithelial keratomileusis (LASEK) refractive eye surgery, or laser-assisted in situ keratomileusis (LASIK) refractive eye surgery.

57. A method for the treatment of pain or inflammation associated with eye surgery, wherein the method comprises:
pre-dosing a patient for a period up to 48 hours before the eye surgery with the ocular formulation of claims 1 to 43, wherein the formulation is applied to an eye of the patient from one to three times daily; or
applying the formulation post-surgery for about 14 days or until the pain or inflammation has been alleviated, wherein the formulation is applied to the eye of the patient from one to three times daily.

58. The method of claim 57, wherein the method further comprises administering the ocular formulation to the eye of the patient before, during, or after the eye surgery.

59. The method of claim 57 or 58, wherein the method comprises both pre-dosing the patient and applying the formulation post-surgery.

60. The method of claim 50, wherein the eye surgery is cataract surgery.

61. The method of claim 50, wherein the pain or inflammation is caused by postoperative eye surgery.

62. The method of claim 50, wherein the pain is associated with refractive surgery.

63. The method of claim 62, wherein the refractive surgery is photorefractive keratectomy (PRK) surgery, laser epithelial keratomileusis (LASEK) refractive eye surgery, or laser-assisted in situ keratomileusis (LASIK) refractive eye surgery.
